(19) United States
(12) Patent Application Publication
(10) Pub. No.: US 2023/0218533 A1
(43) Pub. Date: Jul. 13, 2023

(54) 3D LASER SINTERING PROCESSES FOR IMPROVED DRUG DELIVERY

(71) Applicant: BOARD OF REGENTS, THE UNIVERSITY OF TEXAS SYSTEM, Austin, TX (US)

(72) Inventors: Robert O. WILLIAMS, III, Austin, TX (US); Mohammed MANIRUZZAMAN, Pflugerville, TX (US); Daniel A. DAVIS, JR., Austin, TX (US); Rishi THAKKAR, Austin, TX (US)

(73) Assignee: BOARD OF REGENTS, THE UNIVERSITY OF TEXAS SYSTEM, Austin, TX (US)

(21) Appl. No.: 18/001,411

(22) PCT Filed: Jun. 10, 2021
(86) PCT No.: PCT/US2021/036864

§ 371 Date: Dec. 9, 2022

Related U.S. Application Data
(60) Provisional application No. 63/037,586, filed on Jun. 10, 2020.

(51) Int. Cl. A61K 9/20; A61K 41/00; A61K 31/4422; A61K 31/427; B29C 64/153; B33Y 80/00; B33Y 70/00

(52) U.S. Cl. CPC A61K 9/2095; A61K 41/00; A61K 31/4422; A61K 31/427; A61K 9/2027; A61K 9/2013; A61K 9/2009; B29C 64/153; B33Y 80/00; B33Y 70/00; B29L 2031/753

(57) ABSTRACT

The present disclosure provides pharmaceutical compositions prepared using an additive manufacturing process where the active pharmaceutical ingredient has been rendered into the amorphous form or prepared as an amorphous solid dispersion at a temperature below the melting point of the active pharmaceutical ingredient or the glass transition of the physical mixture or composition of the individual components. The present disclosure also provides methods of preparing these compositions by using properties such as the chamber and surface temperature and the electron laser density.

This application claims the benefit of priority to U.S. Provisional Application No. 63/037,586, filed on Jun. 10, 2020, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition as amorphous solid dispersions through additive manufacturing techniques.

### 2. Description of Related Art

A significant number of molecules developed in the pharmaceutical drug discovery and lead optimization process are eliminated due to their dose-dependent poor water solubility and thereby low bioavailability. Many of the marketed drug substances also suffer from poor aqueous solubility and thereby fall under the class II and IV of the biopharmaceutical classification system (BCS), which means that the highest available dose of the drug is insoluble in 250 mL of simulated gastric/intestinal fluid. The pharmaceutical industry has adapted amorphous solid dispersions (ASDs) as a viable formulation technique to overcome these issues. Thermodynamically, a drug in an amorphous state has a higher chemical potential as compared to the crystalline state and thereby amorphous state has a higher reactivity and it depicts an enhanced solubility than the crystalline state which is relatively more stable. Although pure amorphous drugs have a solubility advantage over the crystalline species, they are extremely unstable because of their enhanced reactivity and hence tend to recrystallize and/or form hydrates and solvates by trapping water or other solvents in their lattice, this might lead to degradation and/or altered therapeutic activity of the drug. Considering this, formulations or processes leading to partial amorphous conversions or suffering recrystallization in the biological system would have an unpredictable release, absorption, and thereby bioavailability. Hence, these type of formulations cannot be considered as a viable pharmaceutical dosage form. This phenomenon of partial amorphous conversion has been observed in multiple selective laser sintering based 3D printed dosage forms, reported previously in the literature. Even though the partial amorphous conversion was observed in these examples, the phenomenon was not intended or controlled by manipulating the processing parameters. Moreover, the state of the amorphous drug was not investigated as to whether it formed an ASD or merely drug in amorphous form.

ASDs stabilize the amorphous drug by dispersing it in the polymeric matrix. This prevents the drug from recrystallizing. Moreover, the polymer controls the release of the amorphous drug from its matrix, this ensures the controlled release of the drug from the polymeric matrix, which prevents the recrystallization of the drug in the biological system. The two most commonly utilized methods for the preparation of ASDs include hot-melt extrusion (HME) and Spray drying (SD), which are used for the majority of drugs, but they have significant limitations. Innovative techniques (thin-film freezing, TFF; KinetiSol Processing, KSD; and micro precipitated bulk powder, MBP) have been developed as viable alternatives to creating ASDs. The solubility advantage of ASD in comparison to the pure crystalline drug has been outlined in various published literature. However, the crystalline state is more stable than the amorphous state. Thereby amorphous conversion of BCS class I & III drugs would impact their release profile and stability upon storage. Previously, the preparation of dosage forms using Selective laser sintering three-dimensional printing (SLS-3DP) has been employed BCS class III drugs such as acetaminophen (APAP) and the final product contains a partial amorphous conversion of the drug. This phenomenon of partial amorphous conversion of BCS class III drugs impacts the release behavior of the formulation, making it unpredictable, and the stability of the formulation is also compromised as the drug molecules become more reactive. In a nutshell, amorphous conversion and formulation of BCS II and IV drugs as ASDs provide stabilized and predictable solubility and bioavailability enhancement, in contrast to partial amorphous conversion, which might lead to unpredictability in release behavior of the formulation and suffer instability on storage.

Advances in amorphous solid dispersion development have emphasized the importance of both mixing and temperature for thermal or thermokinetic processes (e.g., HME, Kinetisol Processing). In these processes conversion to the amorphous state is dependent both on temperature and the degree of mixing. It is less obvious to appreciate the degree of mixing's impact on the conversion of the crystalline to the amorphous state. Mixing both increases the overall diffusion of the system and decreases the diffusion layer thickness, increasing the tendency of the drug to dissolve within the molten polymer. Conversion to the amorphous state significantly below a composition's melting point has been reported for systems that utilize mixing. Hot stage polarized light microscopy (HSPLM) can be used to better understand this phenomenon. In this technique, the drug is sprinkled onto a polymeric film that is then heated to the desired temperature. If the temperature is held constant at a selected temperature below the composition's melting point the crystalline drug fails to be converted to the amorphous state if the selected temperature is below the composition's melting point. In the same system, it has been reported that the API can be converted amorphous by HME or KSD at temperatures substantially below the melting point. For systems that do not utilize mixing (e.g., melt quenching and 3DP-SLS) a temperature at or above the melting point of the API in compositions' melting point ensures complete crystalline conversion to the amorphous phase. At temperatures below this temperature, partially amorphous systems that contain trace crystallinity would be suspected. Trace crystallinity in amorphous solid dispersions acts as a “seed” to promote future crystal growth, compromising stability and altering both the dissolution and bioavailability.

Selective Laser Sintering Three-Dimensional Printing (SLS-3DP) is emerging as a viable method to produce pharmaceutical tablets. Research has been dedicated to showing the dynamic applications of this process to the pharmaceutical fields. Specifically, SLS-3DP has been able to highlight its ability to create patient-tailored medications by modification of the printing parameters. The prior art has shown the ability to control the drug release from the tablet matrix by using the highly precise laser to configure different lattice structures. These structures have the ability to control drug release by altering the surface area of the tablet that is exposed to the media. A combination of different polymers incorporated within the SLS process has shown to be able to control drug release as well. A focus of these works has been on delivery of BCS Class III drugs, specifically acetaminophen (APAP), and the ability to construct different tablets with various polymers along with the ability to modify the release. In those publications, it has been suggested that APAP is rendered slightly amorphous as a byproduct of the SLS product. In another example, the first time a BCS Class II drug, ibuprofen, is incorporated into a SLS product was a fixed-dose combination tablet to show the ability of SLS to incorporate multiple drugs in the printing process. The intention of this study highlights the feasibility of the SLS process to incorporate multiple drugs within the printing process while controlling drug release by modification of the tablet design. It is briefly noted that again the product appears partially amorphous but as a byproduct of the process and not by intentional design. Dissolution was performed on these tablets with no improvement of dry solubility by amorphous conversion or increase in bioavailability.

Prior publication bases their printing parameters dependent on the polymers glass transition temperature or temperature between the Tₘ and Tₘ/2. Pending patent applications have suggested the range of 0-400° C. for surface temperature when printing. Though this range could print a tablet, it would not ensure an amorphous system was created. This relates to the fundamental understanding that processing above the melting point when mixing is absent enables the complete amorphous conversion and eliminates any trace crystallinity. Printing parameters must be intentionally chosen to ensure the polymer will not melt at the surface temperature needed to be slightly below the melting point of the API in composition. When the laser is applied to the system it raises the surface temperature of the composition slightly above the API to induce melting and initiates the complete conversion to the amorphous state. Therefore, a system could benefit from a method of producing a drug product that takes the compositions' melting point to design the system's printing parameters.

Previously the ability to control drug release of both individual and combination drug products by modifying printing parameters of the SLS-3DP has been evaluated. Dissolution studies have shown the ability to control the release but solubility enhancement has not been demonstrated. Understanding that the SLS-3DP process does not involve mixing, printing parameters above the composition's melting point enables conversion to the amorphous state, and a clear benefit of the SLS-3DP ASD is seen during the dissolution process. Furthermore, there remains a need for a system that can achieve solubility enhancement by intentional and complete amorphous conversion of an SLS-3DP printed tablet.

### SUMMARY OF THE INVENTION

The present disclosure provides pharmaceutical compositions that comprise an electromagnetic energy absorbing excipients. Without wishing to be bound by any theory, the present pharmaceutical compositions may result in compositions which are more stable against degradation of the active agent. The active agent may be one that is poorly soluble or maybe one that undergoes chemical degradation after being exposed to heat or shear stress.

In some aspects, the present disclosure provides methods of preparing a pharmaceutical composition comprising:

(A) obtaining a composition comprising:

(1) an active pharmaceutical ingredient;

(2) a pharmaceutically acceptable polymer; and

(3) an electromagnetic energy-absorbing excipient;

(B) sintering the composition using a laser in an additive manufacturing process;
to obtain a pharmaceutical composition, wherein the pharmaceutical composition comprises at least 75% of the active pharmaceutical ingredient in the amorphous form.

In some embodiments, the pharmaceutical compositions comprise at least 90% of the active pharmaceutical ingredient in the amorphous form. In some embodiments, the pharmaceutical compositions comprise at least 95% of the active pharmaceutical ingredient in the amorphous form. In some embodiments, the pharmaceutical compositions comprise at least 99% of the active pharmaceutical ingredient in the amorphous form. In some embodiments the active pharmaceutical ingredient is present in the pharmaceutical composition as an amorphous solid dispersion.

In some embodiments the active pharmaceutical ingredient is a poorly soluble drug. In some embodiments, the active pharmaceutical ingredient is a BCS class 2 drug. In some embodiments, the active pharmaceutical ingredient is a BCS class 3 drug. In some embodiments, the active pharmaceutical ingredient is a BCS class 4 drug. In some embodiments, the active pharmaceutical ingredient is an agent which undergoes degradation at an elevated temperature in a formulation process. In some embodiments, the active pharmaceutical ingredient is chemically sensitive to temperature. In some embodiments, the active pharmaceutical ingredient is chemically sensitive to shear. In some embodiments, the active pharmaceutical ingredient is an agent with a melting point of greater than about 60° C. In some embodiments, the melting point is from about 60° C. to about 300° C. In some embodiments, the melting point is from about 80° C. to about 200° C.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is an anti-viral agent, antibiotic agent, nonsteroidal anti-inflammatory agent, or heat sensitive agent. In some embodiments, the anti-viral agent is an anti-retroviral. In other embodiments, the active pharmaceutical ingredient is an anti-hypertensive agent such as a calcium channel blocker.

In some embodiments, the pharmaceutical compositions comprise from about 1% w/w to about 90% w/w of the active pharmaceutical ingredient. In some embodiments, the pharmaceutical compositions comprise from about 5% w/w to about 50% w/w of the active pharmaceutical ingredient. In some embodiments, the pharmaceutical compositions comprise from about 10% w/w to about 30% w/w of the active pharmaceutical ingredient. In other embodiments, the pharmaceutical composition comprises from about 5% w/w to about 30% w/w of the active pharmaceutical ingredient. In some embodiments, the pharmaceutical composition comprises a ratio of the active pharmaceutical ingredient to the electromagnetic energy-absorbing excipient from about 5:1 to about 1:10. In some embodiments, the ratio is from about 2:1 to about 1:5. In some embodiments, the ratio is from about 1:1 to about 1:3 such as about 1:1, 1:1.5, or 1:3.

In some embodiments, the pharmaceutically acceptable polymer is a cellulosic polymer. In some embodiments, the cellulosic polymer is a neutral cellulosic polymer. In some embodiments, the cellulosic polymer is a charged cellulosic polymer. In some embodiments, the pharmaceutically acceptable polymer is a neutral non-cellulosic polymer. In some embodiments, the neutral non-cellulosic polymer comprises a poly(vinyl acetate), poly(vinylpyrrolidone), poly(ethylene glycol), poly(ethylene oxide), poly(vinyl alcohol), or methacrylate unit. In some embodiments, the pharmaceutically acceptable polymer comprises a poly(vinyl acetate) or a methacrylate unit. In some embodiments, the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

In some embodiments, the pharmaceutical compositions comprise from about 5% w/w to about 95% w/w of the pharmaceutically acceptable polymer. In some embodiments, the pharmaceutical compositions comprise from about 50% w/w to about 90% w/w of the pharmaceutically acceptable polymer. In some embodiments, the pharmaceutical compositions comprise from about 60% w/w to about 90% w/w of the pharmaceutically acceptable polymer.

In some embodiments, the electromagnetic energy-absorbing excipient is a material that leads to improved energy absorption. In some embodiments, the electromagnetic energy-absorbing excipient is a material with a lambda max (λₘₐₓ) equal to the wavelength of the laser. In some embodiments, the lambda max is from about 50 nm to about 15,000 nm. In some embodiments, the lambda max is from about 200 nm to about 11,000 nm. In some embodiments, the lambda max is from about 200 nm to about 1,000 nm.

In some embodiments, the electromagnetic energy-absorbing excipient is an inorganic material. In some embodiments, the electromagnetic energy-absorbing excipient is an aluminum material. In some embodiments, the aluminum material is an aluminum inorganic salt. In some embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In some embodiments, the aluminum inorganic salt is potassium aluminum silicate with a coating of titanium dioxide and/or iron oxide. In some embodiments, the inorganic material is iron oxide, titanium oxide, or silicates. In some embodiments, the electromagnetic energy-absorbing excipient is an organic material. In some embodiments, the organic material is a dye. In some embodiments, the dye is carmine, a phthalocyanine, or a diazo compound.

In some embodiments, the pharmaceutical compositions comprise from about 0.01% w/w to about 60% w/w of the electromagnetic energy-absorbing excipient. In some embodiments, the pharmaceutical compositions comprise from about 0.1% w/w to about 50% w/w of the electromagnetic energy-absorbing excipient. In some embodiments, the pharmaceutical compositions comprise from about 1% w/w to about 30% w/w of the electromagnetic energy-absorbing excipient. In some embodiments, the pharmaceutical compositions comprise from about 1% w/w to about 10% w/w of the electromagnetic energy-absorbing excipient.

In some embodiments, the methods comprise using a laser with sufficient energy to cause the conversion of the active pharmaceutical ingredient to an amorphous form. In some embodiments, the methods comprise exposing the composition to a laser in a pattern. In some embodiments, the pattern is prepared by passing the laser over the composition with a laser speed from about 5 mm/s to about 50,000 mm/s. In some embodiments, the laser speed is from about 10 mm/s to about 1,000 mm/s. In some embodiments, the laser speed is from about 25 mm/s to about 300 mm/s such as from about 200 mm/s to about 300 mm/s. In some embodiments, the laser speed is 50 mm/s, 75 mm/s, or 100 mm/s. In some embodiments, the laser has a hatch spacing from about 5 mm to about 100 mm. In some embodiments, the hatch spacing is from about 10 mm to about 50 mm. In some embodiments, the hatch spacing is from about 10 mm to about 40 mm. In some embodiments, the hatch spacing is about 25 mm. In some embodiments, the laser comprises a laser power from about 0.1 W to about 250 W. In some embodiments, the laser power is from about 0.5 W to about 150 W. In some embodiments, the laser power is from about 1 W to about 100 W. In some embodiments, the laser power is from about 1 W to about 10 W.

In some embodiments, the methods comprise depositing a layer in a chamber. In some embodiments, the layer has a layer thickness from about 1 μm to about 100 mm. In some embodiments, the layer thickness is from about 10 μm to about 10 mm. In some embodiments, the layer thickness is from about 50 μm to about 1 mm. In some embodiments, the layer thickness is from 50 μm to about 100 μm.

In some embodiments, the layer comprises a surface temperature at its surface different from a chamber temperature in the chamber. In some embodiments, the surface temperature is from about 0° C. to about 250° C. In some embodiments, the surface temperature is from about 50° C. to about 175° C. In some embodiments, the surface temperature is from about 75° C. to about 150° C. In some embodiments, the surface temperature is from about 100° C. to about 120° C. In some embodiments, the chamber temperature is from about 25° C. to about 250° C. In some embodiments, the chamber temperature is from about 50° C. to about 200° C. In some embodiments, the chamber temperature is from about 75° C. to about 150° C. In some embodiments, the surface temperature is more than 15° C. less than the melting point of the composition.

In some embodiments, the laser comprises a beam size from about 0.25 μm to about 1 mm. In some embodiments, the beam size is from about 1 μm to about 500 μm. In some embodiments, the beam size is from about 2.5 μm to about 100 μm. In some embodiments, the laser has a wavelength from about 50 nm to about 15,000 nm. In some embodiments, the wavelength is from about 200 nm to about 11,000 nm. In some embodiments, the wavelength is from about 200 nm to about 1,000 nm. In some embodiments, the laser gives the composition an amount of energy equal to an electron laser density from about 2.5 J/mm³ to about 500 J/mm³. In some embodiments, the electron laser density is from about 5 J/mm³ to about 250 J/mm³. In some embodiments, the electron laser density is from about 7.5 J/mm³ to about 50 J/mm³. In some embodiments, the electron laser density is greater than 2.5 J/mm³. In some embodiments, the electron laser density is greater than 5 J/mm³. In some embodiments, the electron laser density is greater than 7.5 J/mm³.

In some embodiments, the compositions further comprise one or more excipients. In some embodiments, the excipient is a processing aid. In some embodiments, the excipient is an op[acifying agent. In some embodiments, the excipient is an excipient which improves the flowability of the composition. In some embodiments, the excipient is a silicon compound. In some embodiments, the excipient is silicon dioxide. In some embodiments, the composition comprises from about 0.1% w/w to about 5% w/w of the excipient. In some embodiments, the composition comprises from about 0.5% w/w to about 2.5% w/w of the excipient. In some embodiments, the composition comprises from about 0.5% w/w to about 1.5% w/w of the excipient.

In some embodiments, the additive manufacturing technique is selective laser sintering. In some embodiments, the additive manufacturing technique converts the pharmaceutical composition into a unit dose. In some embodiments, the unit dose is an oral dosage form such as a tablet.

In still another aspect, the present disclosure provides pharmaceutical composition prepared according to the methods described herein.

In still yet another aspect, the present disclosure provides pharmaceutical compositions comprising:

(A) an active pharmaceutical ingredient;
(B) a pharmaceutically acceptable polymer; and
(C) an electromagnetic energy-absorbing excipient;
wherein the pharmaceutical comprises at least 75% of the active pharmaceutical ingredient in the amorphous form.

In some embodiments, the pharmaceutical compositions comprise at least 90% of the active pharmaceutical ingredient in the amorphous form. In some embodiments, the pharmaceutical compositions comprise at least 95% of the active pharmaceutical ingredient in the amorphous form. In some embodiments, the pharmaceutical compositions comprise at least 99% of the active pharmaceutical ingredient in the amorphous form. In some embodiments the active pharmaceutical ingredient is present in the pharmaceutical composition as an amorphous solid dispersion.

In some embodiments the active pharmaceutical ingredient is a poorly soluble drug. In some embodiments, the active pharmaceutical ingredient is a BCS class 2 drug. In some embodiments, the active pharmaceutical ingredient is a BCS class 3 drug. In some embodiments, the active pharmaceutical ingredient is a BCS class 4 drug. In some embodiments, the active pharmaceutical ingredient is an agent which undergoes degradation at an elevated temperature in a formulation process. In some embodiments, the active pharmaceutical ingredient is chemically sensitive to temperature. In some embodiments, the active pharmaceutical ingredient is chemically sensitive to shear. In some embodiments, the active pharmaceutical ingredient is an agent with a melting point of greater than about 60° C. In some embodiments, the melting point is from about 60° C. to about 300° C. In some embodiments, the melting point is from about 80° C. to about 200° C.

In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives. In some embodiments, the active pharmaceutical ingredient is an anti-viral agent, antibiotic agent, nonsteroidal anti-inflammatory agent, or heat sensitive agent. In some embodiments, the anti-viral agent is an anti-retroviral. In other embodiments, the active pharmaceutical ingredient is an anti-hypertensive agent such as a calcium channel blocker.

In some embodiments, the pharmaceutical compositions comprise from about 1% w/w to about 90% w/w of the active pharmaceutical ingredient. In some embodiments, the pharmaceutical compositions comprise from about 5% w/w to about 50% w/w of the active pharmaceutical ingredient. In some embodiments, the pharmaceutical compositions comprise from about 10% w/w to about 30% w/w of the active pharmaceutical ingredient. In other embodiments, the pharmaceutical composition comprises from about 5% w/w to about 30% w/w of the active pharmaceutical ingredient. In some embodiments, the pharmaceutical composition comprises a ratio of the active pharmaceutical ingredient to the electromagnetic energy-absorbing excipient from about 5:1 to about 1:10. In some embodiments, the ratio is from about 2:1 to about 1:5. In some embodiments, the ratio is from about 1:1 to about 1:3 such as about 1:1, 1:1.5, or 1:3.

In some embodiments, the pharmaceutically acceptable polymer is a cellulosic polymer. In some embodiments, the cellulosic polymer is a neutral cellulosic polymer. In some embodiments, the cellulosic polymer is a charged cellulosic polymer. In some embodiments, the pharmaceutically acceptable polymer is a neutral non-cellulosic polymer. In some embodiments, the neutral non-cellulosic polymer comprises a poly(vinyl acetate), poly(vinylpyrrolidone), poly(ethylene glycol), poly(ethylene oxide), poly(vinyl alcohol), or methacrylate unit. In some embodiments, the pharmaceutically acceptable polymer comprises a poly(vinyl acetate) or a methacrylate unit. In some embodiments, the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

In some embodiments, the pharmaceutical compositions comprise from about 5% w/w to about 95% w/w of the pharmaceutically acceptable polymer. In some embodiments, the pharmaceutical compositions comprise from about 50% w/w to about 90% w/w of the pharmaceutically acceptable polymer. In some embodiments, the pharmaceutical compositions comprise from about 60% w/w to about 90% w/w of the pharmaceutically acceptable polymer.

In some embodiments, the electromagnetic energy-absorbing excipient is a material that leads to improved energy absorption. In some embodiments, the electromagnetic energy-absorbing excipient is a material with a lambda max (λₘₐₓ) equal to the wavelength of the laser. In some embodiments, the lambda max is from about 50 nm to about 15,000 nm. In some embodiments, the lambda max is from about 200 nm to about 11,000 nm. In some embodiments, the lambda max is from about 200 nm to about 1,000 nm.

In some embodiments, the electromagnetic energy-absorbing excipient is an inorganic material. In some embodiments, the electromagnetic energy-absorbing excipient is an aluminum material. In some embodiments, the aluminum material is an aluminum inorganic salt. In some embodiments, the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide. In some embodiments, the aluminum inorganic salt is potassium aluminum silicate with a coating of titanium dioxide and/or iron oxide. In some embodiments, the inorganic material is iron oxide, titanium oxide, or silicates. In some embodiments, the electromagnetic energy-absorbing excipient is an organic material. In some embodiments, the organic material is a dye. In some embodiments, the dye is carmine, a phthalocyanine, or a diazo compound.

In some embodiments, the pharmaceutical compositions comprise from about 0.01% w/w to about 60% w/w of the electromagnetic energy-absorbing excipient. In some embodiments, the pharmaceutical compositions comprise from about 0.1% w/w to about 50% w/w of the electromagnetic energy-absorbing excipient. In some embodiments, the pharmaceutical compositions comprise from about 1% w/w to about 30% w/w of the electromagnetic energy-absorbing excipient. In some embodiments, the pharmaceutical compositions comprise from about 1% w/w to about 10% w/w of the electromagnetic energy-absorbing excipient.

In some embodiments, the pharmaceutical compositions further comprise one or more excipients. In some embodiments, the excipient is a processing aid. In some embodiments, the excipient is an opacifying agent. In some embodiments, the pharmaceutical compositions comprise a flowability excipient. In some embodiments, the flowability excipient is a silicon compound such as silicon dioxide. In some embodiments, the compositions comprise from about 0.1% w/w to about 5% w/w of the flowability excipient. In some embodiments, the compositions comprise from about 0.5% w/w to about 2.5% w/w of the flowability excipient. In some embodiments, the compositions comprise from about 0.5% w/w to about 1.5% w/w of the flowability excipient. In some embodiments, the pharmaceutical composition shows an increase in the dissolved concentration of greater than 5 fold compared to a physical mixture at neutral pH. In some embodiments, the increase in dissolved concentration is greater than 10 fold compared to a physical mixture at neutral pH. In some embodiments, the pharmaceutical compositions have been processed through an additive manufacturing process. In some embodiments, the additive manufacturing process is selective laser sintering 3D printing. In some embodiments, the additive manufacturing process is used to produce a unit dose. In some embodiments, the unit dose is an oral dosage form such as a tablet.

In still another aspect, the present disclosure provides methods of treating or preventing a disease or disorder in a patient comprising administering to the patient in need thereof a therapeutically effective amount of a pharmaceutical composition described herein, wherein the active pharmaceutical ingredient is therapeutically effective for the disease or disorder.

In still yet another aspect, the present disclosure provides pharmaceutical composition comprising:

(A) an active pharmaceutical ingredient; and
(B) an electromagnetic energy-absorbing excipient;
wherein the pharmaceutical comprises at least 75% of the active pharmaceutical ingredient in the amorphous form.

In still another aspect, the present disclosure provides methods of preparing a pharmaceutical composition comprising:

(A) obtaining a composition comprising:

(1) an active pharmaceutical ingredient; and

(2) an electromagnetic energy-absorbing excipient;

(B) sintering the composition using a laser in an additive manufacturing process;
to obtain a pharmaceutical composition, wherein the pharmaceutical composition comprises at least 75% of the active pharmaceutical ingredient in the amorphous form.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows scans from differential scanning calorimetry of the F1-P4-10 composition shown on the leftmost figure. The middle figure is crystalline ritonavir. The rightmost image is the physical mixture of formulation 1 (RTV: Va64: Candurin®).

FIG. 2 shows the powder X-ray diffraction results of the F1-P4-10 composition. The composition exhibits a broad halo except at 25.2 theta-degrees which is attributed to Candurin®. The physical mixture was included to show areas where crystalline ritonavir would be present.

FIG. 3 shows the Fourier transform infrared spectroscopy results of the F1-P4-10 composition. Peaks that are attributed to ritonavir are no longer present within the final composition, suggesting amorphous conversion.

FIG. 4 shows the WAXS XRD determination of the printed material compared to Candurin®. The material appears to not show any crystallinity

FIG. 5 shows the dissolution profile of the physical mixture of the components relative to the SLS 3D printed form which shows the conversion of the materials into an amorphous solid dispersion resulting in a higher concentration of drugs over time.

FIG. 6 shows scans from differential scanning calorimetry of the compositions made as reference examples from US2019037441A1. The reference example indicates the presence of crystallinity and not an amorphous solid dispersion.

FIG. 7 shows the design points in the Box-Behnken design.

FIGS. 8A & 8B show UV-Visible screening studies (FIG. 8A) UV-Visible spectrum of liquid and solid samples from 460-240 nm wavelength (k) (FIG. 8B) increasing absorption with increasing concentration at 400 nm.

FIG. 9 shows the powder X-ray diffraction spectroscopy of screening samples S1-S3 (10% NFD+90% Kollidon® VA64), Physical mixture for screening samples (NFD+Candurin®+Kollidon® VA64), and pure NFD and Candurin® samples. Kollidon® VA 64 was not included as it is known to be amorphous.

FIGS. 10A-10C show high-performance liquid chromatography-Mass spectroscopy isolated and identified (FIG. 10A) nitro derivative-oxidative degradation product (UV exposure) (FIG. 10B) nitroso derivative-photolytic degradation product (visible-light exposure) (FIG. 10C) nifedipine.

FIG. 11 shows the powder X-ray diffraction spectroscopy of DoE samples (Run 1-17), The two-theta (20) values from 20-30 were selected based on the crystalline peaks observed in the physical mixture in FIG. 9. The broken lines represent Candurin® peak at a 20 value of 25 degrees.

FIGS. 12A-12D show the variable-response relationship trends between % Purity and (FIG. 12A) Candurin® (wt %), (FIG. 12B) Surface temperature (° C.), (FIG. 12C) Laser speed (mm/s), (FIG. 12D) All three independent variables.

FIGS. 13A-13C show the contour lines representing constant values of % Purity over variable values of (FIG. 13A) Laser speed and Candurin® (FIG. 13B) Surface temperature and Candurin® (FIG. 13C) Laser speed and Surface temperature.

FIGS. 14A-14E show the variable-response relationship trends between hardness and (FIG. 14A) Candurin® (FIG. 14B) Surface temperature (FIG. 14C) Laser speed (FIG. 14D) 3D surface plot for all three variables (FIG. 14E) Variable-response cube for all three variables.

FIGS. 15A-15D show the 3D response surface plot for (FIG. 15A) Printlet weight against all three variables (FIG. 15B) Printlet density against all three variables. Variable-response cube for (FIG. 15C) Printlet weight against all three variables (FIG. 15D) Printlet density against all three variables.

FIG. 16 shows the differential scanning calorimetry to confirm amorphous conversion in the optimized formulation.

FIG. 17 shows the pH shift in vitro dissolution testing for Run 10, physical mixture, and crystalline NFD. The change in drug concentration at the 35-minute time point is attributed to the dilution of the dissolution medium from 90 mL to 150 mL.

### DESCRIPTION OF ILLUSTRATIVE EMBODIMENTS

In some aspects, the present disclosure relates to methods of using selective laser sintering 3D printing to produce therapeutic drug formulations such as oral formulations such as tablets. Furthermore, the present disclosure also provides pharmaceutical compositions that may be used in these methods to produce drug formulations with selective laser sintering 3D printing. Additionally, these compositions may be used in the treatment or prevention of a disease or disorder that may be treated or prevented by the active pharmaceutical ingredient (API).

Until now it has not been obvious of the potential benefit SLS-3DP can have towards poorly water-soluble drugs, besides printing tablets and controlling drug release, but the present disclosure relates to the ability to convert poorly water-soluble drugs to their amorphous state for solubility and bioavailability improvement. Without wishing to be bound by any theory, it is believed that the importance of hatching spacing and surface temperature in relation to the composition's melting point plays in the ability of SLS-3DP to create a fully amorphous product. The use of several known methods to produce selective laser sintering 3D printing failed to produce an amorphous solid dispersion. Several printing parameters not previously investigated (e.g., hatching spacing) were determined to be relevant printing parameters to create an amorphous solid dispersion. Before adjusting the hatching spacing and other unexplored printing parameters, printing an SLS-3DP amorphous solid dispersion had not been shown by merely adjusting the laser speed, chamber temperature, and surface temperature. This disclosure provides the methods that may be used to obtain an amorphous solid dispersion using 3DP-SLS. The pharmaceutical compositions were created that improved the solubility of the API.

The present disclosure relates to a process developed to manufacture amorphous solid dispersions or amorphous solid dispersion based pharmaceutical dosage forms using selective laser sintering (3D printing platform) for enhancing the solubility of poorly water-soluble drugs, such as BCS class II and BCS class IV. Furthermore, the present disclosure relates to processes wherein the poorly soluble crystalline drug and a polymer are physically blended with or without other processing aids such as binders, fillers, glidants, lubricants, laser absorbing agents, or other pharmaceutical aids. In the present methods, this physical blend is transferred to the reservoir chamber of the SLS based 3D printer, from this reservoir chamber sufficient blend is withdrawn to form one layer in the build chamber which is exposed to the laser, this process is repeated until the 3D structure design fed to the software is manufactured. Under specific printing conditions and component ratios discussed herein, the physical blend exposed to this process can be converted into an amorphous solid dispersion or an amorphous solid dispersion based 3D printed pharmaceutical dosage form. The process described herein may be used as a one-step manufacturing platform for producing amorphous solid dispersions or pharmaceutical dosage forms which may exhibit an enhanced dissolution rate. For example, the methods and pharmaceutical compositions described herein may be used in the manufacturing of amorphous solid dispersions, screening of potential amorphous solid dispersions and their performance, and printing of pharmaceutical dosage forms on-demand for patient-specific therapies and personalized medicine.

For the purposes of exemplifying the methods described herein, compositions comprising an active pharmaceutical ingredient and a polymer that solubilizes or fuses with the API under specific processing conditions to form an amorphous solid dispersion were explored. As a model system, Ritonavir, which is a poorly water-soluble, weakly basic anti-retroviral protease inhibitor used for the treatment of human immunodeficiency virus (HIV), was mixed with Kollidon® VA 64 (copovidone) which is a vinylpyrrolidone-vinyl acetate copolymer in different ratios varying from 5:95 to 30:70. After an experimental study, with this specific drug, a drug percent of more than 30% in the physical blend does not lead to the complete conversion of the blend into an amorphous solid dispersion, but with other APIs, it is believed that a higher drug loading may be achieved depending on the solubility of the drug in the polymer. This ratio of drug loading and polymer depends on the solubilization capacity of the polymer, which is its ability of the polymer to stabilize the drug into its amorphous form. Such stabilization may be determined by tools for thermal analysis such as differential scanning calorimetry (DSC) or thermogravimetric analysis (TGA) to evaluate the compatibility of the drug and the polymer. The drug load is one element that may be considered useful when developing an amorphous solid dispersion using the methods described herein.

One element of the present methods is a surface temperature of the printing process that has been set about 5 to about 50 degrees below the melting temperature of the polymer and the API depending on the thermal event such as the glass transition temperature or melting temperature responsible for the solubilization or fusion of the drug in the polymer. The thermal event can be determined by using theoretical methods such as thermodynamic Flory-Huggins modeling or by using theoretical solubility parameters. The temperature, either glass transition temperature or melting temperature may also be predicted by using experimental thermal techniques such as differential scanning calorimetry or thermogravimetric analysis. The surface temperature for the methods can be defined as the temperature of the layer exposed to the laser before sintering. The surface temperature can be set and controlled using the heat source placed directly above the print bed. Such heat sources include an infrared heating lamp or an inductive heating source. Surface temperature, as used herein, may be defined as the temperature of the composition. This temperature of the composition for the print layer and represents a threshold temperature that when exposed to a laser source traveling at a specific hatching spacing and speed leads to the formation of amorphous solid dispersions. Using this temperature and adjusted laser parameters, the methods led to the complete amorphous conversion of the physical blend.

In the methods described herein, the methods comprise a chamber temperature during the additive manufacturing process set about 5 to about 50 degrees below the surface temperature. This temperature is also, alternatively, below the glass transition temperature of the polymer in the composition. By way of example, the compositions with Ritonavir had the chamber temperature 15 degrees below the surface temperature. As used herein, the chamber temperature for this disclosure can be defined as a temperature of the build chamber that encases the printing surface. The chamber temperature may be used to aid the temperature increment to the surface temperature but at which no thermal events can occur or be escalated in the physical blend in the reservoir chamber or the print chamber. If the chamber temperature close to the surface temperature or close to any thermal events of any of the components in the physical blend can lead to a print failure due to poor flow of the physical blend, the higher chamber temperature could lead to unwanted melt fusion and agglomeration of the drug and the polymer particles in the reservoir bed and the build chamber. Without wishing to be bound by any theory, the chamber temperature should be controlled with respect to the print time for one layer, for example, the longer the print time the chamber temperature should be set to a temperature further from the thermal event, such as the glass temperature or the melting temperature, or the surface temperature, the chamber temperature should be to prevent print failure. While high chamber temperature can lead to poor flow of the physical blend from the reservoir chamber to the build chamber, prolonged exposure to a high surface temperature can lead to the components to fuse together in the chamber bed; it can also lead to temperature based amorphous conversion of the API instead of laser-based amorphous conversion that has certain disadvantages noted above.

Another element of the process is the hatch spacing or hatch distance (HS), which was set to 25 in the present methods. As used herein, the HS may be defined as the minimum distance between the center of one laser beam to the center of the next laser beam as the laser passes over the chamber to print the pharmaceutical composition and thus may be used to convert the physical blend into an amorphous solid dispersion. For the composition described herein, the compositions that had a hatch spacing more than 25 may leave traces of crystallinity in the produced ASD. This particular parameter was used in the present methods in that it allows the laser to travel across the physical mixture in the print bed in a close-knit pattern which ensures the exposure of the laser to the complete print surface. When the HS was increased, the compositions were observed that the physical blend was not entirely fused and forms a brittle, agglomerated mass of powder which exhibits crystallinity. On the other hand, a low HS along with a low laser speed may be used to maintain high levels of area-related energy densities that results in the formation of ASDs. Without wishing to be bound by any theory, it is believed that the HS is closely related to the laser speed and both these parameters along with the print surface area together determine the print time for each layer where the print time is directly proportional to the surface area and inversely proportional to the HS and the laser speed.

In some embodiments, the laser speed (LS) during the printing process was set within the range of about 25 to about 100 mm/sec. As used herein, the laser speed may be defined as the travel speed of the laser or the exposure time of the laser onto the print surface. This speed should be sufficient for the melt solubilization or melt fusion of the components in the physical blend leading to the formation of amorphous solid dispersion. The lower the laser speed the higher the time required to sinter one layer. During successful printing and complete amorphous conversion, a lower laser speed was used. Furthermore, it was also determined that when the laser speed is reduced the surface temperature should also be reduced as a low laser speed and a high surface temperature leads to a print failure. Without wishing to be bound by any theory, it is believed that prolonged exposure of heat to the surface layer leads to print failure. This particular parameter is useful for obtaining an amorphous composition.

The LS and the HS along with the power of the laser and the thickness of the layer provide the volume related electron laser density. Although this equation provides a good approximation regarding the relationship between the mentioned parameters, it does not take into account several materials associated factors. This equation can provide the density of the laser is exposed over a certain volume but the fraction of the energy absorbed for the melt fusion and solubilization of the physical blend to form an ASD is material specific. In some aspects, the energy input into the system by the laser as the electron laser density may also take into consideration other factors such as surface temperature, chamber temperature, drug load, and formulation components.

$$Electron Laser Density ( \frac{J}{{mm}^{3}} ) = \frac{Laser Power ( w )}{LS \times HS \times LT}$$

Additionally, different energy thresholds are needed to print a tablet as well as simply printing a tablet that is in the amorphous state. The total energy applied to the system is a function of the electron laser density, which is defined by the equation above, and the ability of the composition to absorb a percentage of the energy emitted by the laser. Each composition will have a different electron laser density necessary to overcome each threshold dependent on the composition's capacity to absorb at the wavelength emitted by the laser. Previously, the threshold needed to print an SLS-3DP tablet has been explored but such methods had not been able to reach a threshold to print an SLS-3DP tablet wherein the active pharmaceutical ingredient is in the amorphous form. The most comprehensive report of printing parameters were disclosed within U.S. Patent Application No. 2019/037441. This application contains a list but has no mention of hatch spacing. The list includes the following parameters including a surface temperature 0-200° C. preferably 70-170° C., chamber temperature 25-200° C. preferably 60-150° C., layer thickness 10 mm-0.01 mm, beam size 0.0025-1 mm, scan speed 5 mm/s to 50,000 mm/s preferably 20-300 mm/s, Laser power 0.5 W to 140 W preferably 1.7-8 W, and wavelength 200 nm to 11,000 nm. This patent application describes that the Andrew number for each composition should retain a similar value by modification of either the scan speed or the laser power. This number applied to a composition is believed to influence the release properties of a formulation. It has not been suggested that by combining the electron laser density with a composition's ability to absorb electromagnetic radiation at the emitted wavelength a model can be created the total energy absorbed by the composition to determine the increase in temperature as a result of the laser. Tailoring the surface temperature to the maximum temperature without altering the flow properties enables a successful print in combination with using the minimal energy to overcome the melting point of the drug in composition minimizes the potential degradation that could be induced by the laser. Without wishing to be bound by any theory, it is believed that the combination of the electron laser density, absorption of the composition, hatch spacing and that SLS-3DP does not involve mixing allows the user to design a system that uses the laser energy in combination with surface temperature to create an amorphous 3D-printed tablet.

In some aspects, the present disclosure relates to the preparation of amorphous solid dispersion. While ASDs may be prepared using a variety of different processing methods, not all amorphous solid dispersions are created equal. While this fact may seem counterintuitive, as all amorphous solid dispersions experience solubility enhancement, appear amorphous via characterization techniques, and even ssNMR seems to produce similar domain sizes with different processes. Recently, despite the similarity at a molecular level, differences in ASD performance may be attributed to specific characteristics that are process dependent. For example, the preparation of an ASDs prepared using spray drying, the small particle size produced from the product corresponds to an increased surface area of the particle.

Consequently, the differences in the formulation of the ASDs result in greater drug exposure on the surface, promoting a higher tendency to recrystallize upon storage and rapid drug release for enteric dosage, which is not desired. The discrepancy between amorphous products depends on the amount of drug-exposed on the surface. Stability, SEM (porosity), dissolution, and XPS would be viable tests to differentiate differences between ASD by different processes. These differences come down to how well the API is protected and stabilized by the carrier in which it is processed, the more protected, the lower the tendency to crystalize and release quickly upon dissolution. Therefore, the preparation of ASDs through new methodologies and with new processes is important to developing better and more effective ASDs.

### I. PHARMACEUTICAL COMPOSITIONS

In some aspects, the present disclosure provides pharmaceutical compositions containing an active pharmaceutical ingredient or a pharmaceutically acceptable salt, ester, derivative, analog, pro-drug, or solvates thereof, a pharmaceutically acceptable polymer including polymeric excipients, and electromagnetic energy-absorbing excipient such as an inorganic or organic compound that absorbs electromagnetic energy. These compositions may be amorphous in nature and formulated as an amorphous solid dispersion. In some aspects, the pharmaceutically acceptable polymer and the electromagnetic energy-absorbing excipient may be processed to obtain a compound excipient which is then formulated with the active pharmaceutical ingredient. In some embodiments, the pharmaceutical composition is substantially, essentially, or entirely free of any other compound.

In some aspects, the present composition may be substantially, essentially, or entirely free from any plasticizer or similar agents which interact with the pharmaceutical composition on the molecular level. Without wishing to be bound by any theory, it is believed that the electromagnetic energy absorbing excipientsdoes not interact with the pharmaceutical composition but rather acts to facilitate the transfer of heat more efficiently.

Additionally, the present compositions may be converted into an amorphous form at a temperature below the melting point of the active pharmaceutical ingredient or below the glass transition temperature of the composition. This temperature below the melting point of the active pharmaceutical ingredient or the glass transition temperature of the composition may also be referred to as the thermal event. The temperature at which the composition is converted into the amorphous form or into an amorphous solid dispersion is the surface temperature and maybe at least about 1° C., at least about 5° C., at least about 10° C., at least about 15° C., at least about 20° C., at least about 25° C., at least about 30° C., at least about 35° C., at least about 40° C., or at least about 50° C. below the melting point of the active pharmaceutical ingredient or the glass transition temperature. In some embodiments, the methods used herein comprise using heating the composition to a temperature that is from about 1° C. to about 50° C., from about 5° C. to about 40° C., or from about 10° C. to about 30° C. less than the melting point of the active pharmaceutical ingredient or the glass transition temperature. In some embodiments, the pharmaceutical composition comprises at least 75%, at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.8%, or at least 99.9% of the active pharmaceutical ingredient in the amorphous form.

A. Active Pharmaceutical Ingredient

The pharmaceutical compositions described herein comprise an active pharmaceutical ingredient. The pharmaceutical compositions described herein contain an active pharmaceutical ingredient in an amount between about 5% to about 95% w/w, between about 10% to about 90% w/w, between about 10% to about 50% w/w, or between about 10% to about 40% w/w of the total composition. In some embodiments, the amount of the active pharmaceutical ingredient is from about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33% 34%, 35%, 36%, 37%, 38%, 39%, 40%, 42%, 44%, 45%, 46%, 48%, 50%, 52%, 54%, 55%, 56%, 58%, 60%, 65%, 70%, 75%, 80%, to about 90% w/w or any range derivable therein. In some embodiments, the pharmaceutical composition is substantially, essentially, or entirely free of any other active pharmaceutical ingredient. In some embodiments, the pharmaceutical compositions may have a ratio of the of the active pharmaceutical ingredient to the electromagnetic energy-absorbing excipient from about 5:1 to about 1:10, from about 2:1 to about 1:5, or from about 1:1 to about 1:3. The ratio may be 5:1, 4:1, 3:1, 2:1, 1.5:1, 1:1, 1:1.5, 1:2, 1:3, 1:4, 1:5, 1:6, 1:8, or 1:10, or any range derivable therein.

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I—High Permeability, High Solubility; Class II—High Permeability, Low Solubility; Class III—Low Permeability, High Solubility; and Class IV—Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconazole, Lopinavir, Mebendazole, Nelfinavir, Nevirapine, Niclosamide, Praziquantel, Pyrantel, Pyrimethamine, Quinine, and Ritonavir. Antineoplastic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin. Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenytoin, Quetiapine Risperidone, Rofecoxib, and Valproic acid. Dermatological Agents such as Isotretinoin—Endocrine and Metabolic Agents such as Dexamethasone, Danazol, Epalrestat, Gliclazide, Glimepiride, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, pyridostigmine, sodium cloxacillin, thiamine, benzimidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone, BI-639667, rifabutin, paclitaxel, curcumin, etoposide, neomycin, methotrexate, atazanavir sulfate, Aprepitant, amphotericin B, amiodarone hydrochloride, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other active pharmaceutical ingredients that are of specific consideration are those are those that are high melting point drugs such as a drug that has a melting point of greater than 60° C. Alternatively, the active pharmaceutical ingredients used herein may have a melting point from about 35° C. to about 1,000° C., from about 50° C. to about 750° C., or from about 60° C. to about 200° C. In particular, the melting point may be greater than 25° C., 35° C., 50° C., 60° C., 80° C., 100° C., 125° C., 150° C., 175° C., 200° C., or 250° C.

In some aspects, the present methods may be used to formulate one or more poorly soluble active pharmaceutical ingredients such as deferasirox, etravirine, indomethacin, posaconazole, and ritonavir. Etravirine is a neutral active agent and may be used as a model for other neutral active agents. Deferasirox and indomethacin is a weak acid API and may be used as a model for other weak acid APIs. Posaconazole, itraconazole, and ritonavir are weak base APIs and may be used as models for other weak base APIs.

Suitable active pharmaceutical ingredients may be any poorly water-soluble, biologically active pharmaceutical ingredients or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the active pharmaceutical ingredients may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the active pharmaceutical ingredients may be busulfan, taxane, or other anticancer agents; alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole, and c) thiazoles such as abafungin. Other active pharmaceutical ingredients that may be used with this approach include, but are not limited to, hyperthyroid drugs such as carbimazole, anticancer agents like cytotoxic agents such as epipodophyllotoxin derivatives, taxanes, bleomycin, anthracyclines, as well as platinum compounds and camptothecin analogs. The following active pharmaceutical ingredients may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The active pharmaceutical ingredients may also include a psychiatric agent such as an antipsychotic, anti-depressive agent, or analgesic and/or tranquilizing agents such as benzodiazepines. The active pharmaceutical ingredients may also include a consciousness level-altering agent or an anesthetic agent, such as propofol. The present compositions and the methods of making them may be used to prepare a pharmaceutical composition with the appropriate pharmacokinetic properties for use as therapeutics.

In some aspects, the method may be mostly used with active pharmaceutical ingredients which undergo degradation at an elevated temperature or pressure/shear. The active pharmaceutical ingredients that may be used include those which decompose at a temperature above about 50° C. In some embodiments, the active pharmaceutical ingredients decompose above a temperature of 80° C. In some embodiments, the active pharmaceutical ingredients decompose above a temperature of 100° C. In some embodiments, the active pharmaceutical ingredients decompose above a temperature of 150° C. The active pharmaceutical ingredients that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

Alternatively, active pharmaceutical ingredients may be one that is sensitive to shear. These active pharmaceutical ingredients are compounds for which the chemical and/or physical properties may change due to friction resulting from the manufacturing process itself, including chemical degradation of a drug or the loss of molecular weight of a polymer as non-limiting examples. The degree of loss of the chemical or physical properties of a compound due to shear is often seen as a function of the degree of mixing (e.g., blade RPM, rotation speed) and the properties of the polymer carrier (e.g. rheological properties).

B. Excipients

In some aspects, the present disclosure comprises one or more excipients formulated into pharmaceutical compositions including a pharmaceutically acceptable polymer and an electromagnetic energy absorbing excipients. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, opacifying agent, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

1. Electromagnetic Energy Absorbing Excipients

In some aspects, the pharmaceutical composition may further comprise one or more inorganic or organic material that promotes the absorbance of electromagnetic energy. In one embodiment, the electromagnetic energy-absorbing excipient is inert and does not interact with the formulation. Without wishing to be bound by any theory, it is believed that the addition of the electromagnetic energy-absorbing excipient increases the ability of the system to readily disperse energy throughout the formulation. By increasing the efficiency of electromagnetic energy when exposed to a laser, it is believed that the addition eliminates the total amount of energy needed to cover the composition into an amorphous form. The addition of these materials thus may be used to create a more favorable formation of an amorphous material such as an amorphous solid dispersion.

In some embodiments, the pharmaceutical compositions of the present disclosure include one or more inorganic and/or organic materials as the electromagnetic energy-absorbing excipient. Some non-limiting examples of electromagnetic energy-absorbing excipient (EEAE) include: Candurin® (potassium aluminum silicate (mica) with a coating of Titanium dioxide and/or iron oxide), Potassium aluminum silicate (PAS), aluminum, aluminum sulfates, sodium aluminum phosphate acidic, sodium aluminum silicate, calcium aluminum silicate, bentonite, starch aluminum octenyl succinate and other aluminum consisting composition. A skilled artisan would be aware of such aluminum based EEAEs which may be used in the pharmaceutical compositions described herein. In some embodiments, the EEAEs may absorb energy at a lambda max from about 50 nm to about 15,000 nm, from about 100 nm to about 11,000 nm, from about 200 nm to about 1,100 nm, or from about 250 nm to about 900 nm. In some embodiments, the energy is from a laser with a lambda max from about 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Some other non-limiting examples of inorganic electromagnetic energy-absorbing excipients that may be used include iron oxide, titanium oxide, silicates. In other embodiments, the EEAE may be an organic material, such as a dye. Some non-limiting examples of dyes which may be used include carmine, phthalocyanine, and diazos.

In some embodiments, the EEAE is a compound or composition that is already an FDA approved excipient for human consumption. One example of an EEAE that is approved for human consumption and may be incorporated within the pharmaceutical composition is Candurin®. Candurin® is not soluble in water or other biorelevant conditions making it not be completely digested upon consumption but rather only subject to extraction by stomach acids. Candurin® and other aluminum derivatives are often used as a commercially available food additive in confections, candy, decorations, and beverages at maximum concentrations of 1.25%, equating to a range of 10 mg/kg-323 mg/kg/day. Candurin® contains pearlescent pigments achieve their different coloring effects by using different degrees of titanium oxide and/or iron oxide around a potassium aluminum silicate (PAS) core. The pearlescent color effect results from the partial transmittance and partial reflection of light as well as interference of light through the platelets. PAS-BPP comes in three types all types (types I-III) and may be used in this application. In particular, it is noted that PAS-BPP is expected to have excellent thermal stability during food processing and storage, as the thermal conditions experienced are mild in comparison to which the PAS-BPP is made (900 degree Celsius). Therefore, any Candurin® may be used in this application.

Furthermore, the pharmaceutical composition described herein have a concentration of the electromagnetic energy-absorbing excipient ranging from about 0.01% to about 80% w/w. In some embodiments, the amount of electromagnetic energy-absorbing excipient is from about 0.1% to about 60% w/w, from about 0.5% to about 50% w/w, 1% to about 40% w/w, 1% to about 15% w/w, or 2% to about 10% w/w, wherein the weight is measured against the entire composition weight. The amount of electromagnetic energy-absorbing excipient may be from about 1%, 2%, 3%, 4%, 5%, 6%, 7%, 8%, 9%, 10%, 15%, 20%, 25%, 30%, 40%, 50%, 60%, 70%, to about 80%, or any range derivable therein. In some embodiments, the pharmaceutical composition is substantially, essentially, or entirely free of any other electromagnetic energy-absorbing excipient.

2. Pharmaceutically Acceptable Polymers

In some aspects, the present disclosure provides compositions which may further comprise a pharmaceutically acceptable polymer. In some embodiments, the polymer (polymer carrier) has been approved for use in a pharmaceutical formulation and is known to undergo softening or increased pliability when raised above a specific temperature without substantially degrading.

When a pharmaceutically acceptable polymer is present in the composition, the pharmaceutically acceptable polymer is present in the composition at a level between 1% to 90% w/w, between 10% to 80% w/w, between 20% to 70% w/w, between 30% to 70% w/w, between 40% to 60% w/w. In some embodiments, the amount of the pharmaceutically acceptable polymer is from about 5%, 10%, 15%, 50%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, to about 90% w/w or any range derivable therein. In some embodiments, the pharmaceutical composition is substantially, essentially, or entirely free of any other pharmaceutically acceptable polymer.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, and poloxamer. Within this class, in some embodiments, pyrrolidone containing polymers are particularly useful. Some non-limiting examples of charged cellulosic polymers include cellulose acetate phthalate and hydroxypropyl methylcellulose acetate succinate. Finally, some non-limiting examples of neutral cellulosic polymers include hydroxypropyl cellulose, hydroxypropyl methylcellulose, hydroxyethylcellulose, and hydroxymethyl cellulose.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

Additional pharmaceutically acceptable polymers that may be used in the presently disclosed pharmaceutical compositions include but are not limited to polyethylene oxide; polypropylene oxide; polyvinylpyrrolidone; polyvinylpyrrolidone-co-vinyl acetate; acrylate and methacrylate copolymers; polyethylene; polycaprolactone; polyethylene-co-polypropylene; alkyl celluloses such as methylcellulose; hydroxyalkyl celluloses such as hydroxymethyl cellulose, hydroxyethylcellulose, hydroxypropyl cellulose, and hydroxy butyl cellulose; hydroxyalkyl alkyl celluloses such as hydroxyethyl methylcellulose and hydroxypropyl methylcellulose; starches, pectins; polysaccharides such as tragacanth, gum arabic, guar gum, and xanthan gum. One embodiment of the pharmaceutically acceptable polymer is poly(ethylene oxide) (PEO), which can be purchased commercially from companies such as the Dow Chemical Company, which markets PEO under the POLY OX@ exemplary grades of which can include WSR N80 having an average molecular weight of about 200,000; 1,000,000; and 2,000,000. 3. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active agent in a given solution so that the particles formed contain more than one active agent. In particular, the compositions may further comprise one or more flowability excipients such as a silicon compound. The silicon compound may include an oxide of silicon such as silicon dioxide.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active agent in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include: sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol. In particular, the composition may further comprise one or more silicon compounds such as silicon dioxide that improves the flowability of the composition.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In other aspects, the present compositions may further comprise one or more opacifying agents which modulate the amount of energy absorbed by the composition. Opacifying agents include such compounds as titanium oxide and alter the clarity and ability of electromagnetic energy to be absorbed by the compositions. Alternatively, these compositions may alter the amount of energy needed to achieve appropriate processing of the compositions. Some non-limiting examples of opacifying agents include those taught by U.S. Pat. Nos. 4,009,139, 5,571,334, and PCT Patent Application No. WO 2020/122950, the entire contents of which are hereby incorporated by reference. Some non-limiting examples of opacifying agents including Aerosil®, Cab-O Si®, or other silicon dioxides, aluminum hydroxide, alumina, aluminum silicate, arachidic acid, barium sulfate, bentonite, calamine, calcium carbonate, calcium phosphate dibasic, calcium phosphate tribasic, calcium silicate, calcium sulfate, ceric oxide, cetyl alcohol, activated charcoal, charcoal, diatomaceous earth, erucamide, ethylene glycol monosterate, Fuller's earth, guanine, hectorite, kaolin, magnesium aluminum silicate, magnesium carbonate, magnesium oxide, magnesium phosphate tribasic, magnesium silicate, magnesium trisilicate, myristic acid, palmitic acid, silica, stannic oxide, stearic acid amide, stearoyl monoethanolamine sterate, stearyl palmitate, talc, titanium dioxide, Veegum® or other granular magnesium aluminum silicates, zinc carbonate basic, zirconium oxide, or zirconium silicate.

In some aspects, the amount of the excipient in the pharmaceutical composition is from about 0.1% to about 20% w/w, from about 0.25% to about 10% w/w, from about 0.5% to about 7.5% w/w, or from about 0.5% to about 5% w/w. The amount of the excipient in the pharmaceutical composition comprises from about 0.1%, 0.2%, 0.25%, 0.3%, 0.4%, 0.5%, 0.6%, 0.7%, 0.75%, 0.8%, 0.9%, 1%, 1.25%, 1.5%, 1.5%, 1.75%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 0.25% to 2.5% w/w of the total weight of the pharmaceutical composition.

### II. ADDITIVE MANUFACTURING METHODS

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

In other aspects, the pharmaceutical compositions described herein may also be used in an additive manufacturing platform. Some of the additive manufacturing platforms that may be used herein include 3D printing such as selective laser sintering or selective laser melting. Alternatively, a method such as stereolithography or fused deposition modeling may be used to obtain the final pharmaceutical composition.

These pharmaceutical compositions may be processed through laser sintering wherein a laser is aimed at a specific point on the pharmaceutical composition such that material is bound together to create a solid form. The laser is passed over the surface in a sufficient amount of time and sufficient location to produce the desired dosage form. The method relates to the use of the laser-based upon the power of the laser such as the peak laser power rather than the laser duration. The method often will make use of a pulsed laser. The laser used in these methods often is a high power laser such as a carbon dioxide laser. The process builds up the dosage form using cross-sections of the material through multiple scanning passes over the material. Additionally, the chamber of the 3D printer device may also be preheated to a temperature just below the melting point of the pharmaceutical composition such as the melting point of the composition as a whole or the active pharmaceutical ingredient, the pharmaceutically acceptable polymer, or the combination. Furthermore, the method may be used without the need for a secondary feeder of material into the chamber of the device.

In some embodiments, the additive manufacturing techniques used in the present methods may include selective laser sintering 3D printing. This method may comprise use of a laser onto a composition that has been deposited into a chamber at particular locations. The laser acts to sinter the composition into an amorphous form that may be used as a pharmaceutical composition. The formation of the final product is based upon the energy of the laser as well as the properties of the composition and the temperature of the composition and the chamber that the compositions are deposited into.

In the first part of the selective laser sintering process, the composition is deposited onto a surface in the chamber. The deposition of the composition may result in a layer, wherein the layer of the composition has a layer thickness (LT) from about 0.1 μm to about 100 mm, from about 1 μm to about 100 mm, from about 10 μm to about 100 mm, from about 50 μm to about 10 mm, from about 50 μm to about 1 mm, or from about 50 μm to about 100 μm. The layer thickness may be from about 0.1 μm, 1 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 175 μm, 200 μm, 250 μm, 300 μm, 350 μm, 400 μm, 450 μm, 500 μm, 600 μm, 700 μm, 750 μm, 800 μm, 900 μm, 1 mm, 5 mm, 10 mm, 25 mm, 50 mm, 75 mm, to about 100 mm.

The composition deposited into the surface in the chamber may be heated to a temperature, known as the surface temperature. This surface temperature may be used to provide additional energy to the composition to assist the conversion of the active pharmaceutical ingredient. The surface temperature may be a temperature form about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable.

Furthermore, the chamber may also be heated to a temperature known as the chamber temperature. The chamber temperature may be a temperature form about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the chamber temperature is at least 1° C., at least 5° C., at least 10° C., at least 15° C., at least 20° C., at least 25° C., or at least 50° C. less than the surface temperature. The chamber temperature may be from 1° C. to about 50° C., 5° C. to about 25° C., 10° C. to about 25° C., or 10° C. to about 20° C. less than the surface temperature.

Once the composition has been deposited therein, the composition is exposed to a laser to sinter the composition to obtain the final pharmaceutical composition. The parameters of the laser may be used in obtaining an amorphous composition from the composition deposited in the chamber. The particular laser used by the process may further comprise a laser power from about 0.1 W to about 250 W, from about 0.5 W to about 150 W, from about 1 W to about 100 W, or from about 1 W to about 10 W. The laser used herein may have a laser power from about 0.1 W, 0.5 W, 1 W, 2 W, 3 W, 4 W, 5 W, 6 W, 7 W, 8 W, 9 W, 10 W, 15 W, 20 W, 25 W, 30 W, 35 W, 40 W, 45 W, 50 W, 60 W, 70 W, 80 W, 90 W, 100 W, 125 W, 150 W, 200 W, to about 250 W, or any range derivable therein. The particular laser used may include a high power laser such as carbon dioxide laser, lamp or diode, pumped ND:YAG laser, and disk or fiber lasers. In some embodiment, a 2.3 watt solid diode 455 nm wavelength (visible light, bright blue) laser may be used. The laser used may emit light with a wavelength from about 50 nm to about 15,000 nm, from about 200 nm to about 11,000 nm, or from about 200 nm to about 1,000 nm. The wavelength may be 50 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Furthermore, the laser used may have a specific beam size that indicates the size of the laser that strikes any particular point of the composition at a given time. The methods may further comprise using a laser with a beam size from about 0.1 μm to about 10 mm, from about 0.25 μm to about 1 mm, from about 1 μm to about 500 μm, or from about 2.5 μm to about 100 μm. The beam size may be a size from about 0.1 μm, 0.5 μm, 1 μm, 2.5 μm, 5 μm, 7.5 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 250 μm, 500 μm, 750 μm, 1 mm, to about 5 mm, or any range derivable therein.

The laser may be used to sinter the composition in a pattern. During the sintering process, the laser traces a pattern over the composition to prepare the final pharmaceutical composition. The pattern is prepared by passing the laser over the composition at a specific speed known as the laser speed (LS). The laser speed may be from about 1 mm/s to about 100,000 mm/s, from about 5 mm/s to about 50,000 mm/s, from about 10 mm/s to about 1,000 mm/s, or from about 25 mm/s to about 250 mm/s. The laser speed may be from about 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. Furthermore, the laser may pass in a pattern over the composition in the surface of the chamber. The distances between the lines in the laser's pass are known as hatches. The distance between each successive laser pass is known as the hatch spacing. The methods used herein may include using a hatch spacing from about 5 mm to about 100 mm, from about 10 mm to about 75 nm, from about 10 mm to about 50 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, to about 100 mm, or any range derivable therein.

Finally, the combination of the chamber temperature and the surface temperature may be used to combine with the laser energy to provide sufficient energy to obtain an amorphous active pharmaceutical ingredient. The amount of energy that the laser imparts into the pharmaceutical composition is calculated as the electron laser density. Electron laser density may be calculated using the following formula:

$$Electron Laser Density ( \frac{J}{{mm}^{3}} ) = \frac{Laser Power ( w )}{LS \times HS \times LT}$$

The electron laser density may be an amount of energy imparted from the laser from about 1 J/mm³ to about 500 J/mm³, from about 2.5 J/mm³ to about 500 J/mm³, from about 5 J/mm³ to about 250 J/mm³, from about 7.5 J/mm³ to about 100 J/mm³, or from about 7.5 J/mm³ to about 50 J/mm³. The electron laser density is from about 1 J/mm³, 1.5 J/mm³, 2 J/mm³, 2.5 J/mm³, 3 J/mm³, 3.5 J/mm³, 4 J/mm³, 4.5 J/mm³, 5 J/mm³, 5.5 J/mm³, 6 J/mm³, 6.5 J/mm³, 7 J/m³, 7.5 J/mm³, 8 J/mm³, 8.5 J/mm³, 9 J/mm³, 9.5 J/mm³, 10 J/mm³, 12.5 J/mm³, 15 J/mm³, 17.5 J/mm³, 20 J/mm³, 25 J/mm³, 50 J/mm³, 75 J/mm³, 100 J/mm³, 150 J/mm³, 200 J/mm³, 250 J/mm³, 300 J/mm³, 400 J/mm³, to about 500 J/mm³, or any range derivable therein.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active pharmaceutical ingredient”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “degradation” or “chemically sensitive” refers to a compound that is destroyed or rendered inactive and unacceptable for use. Degradation may include compounds which have one or more chemical bonds present in the compound has been broken.

The term “dissolution” as used herein refers to a process by which a solid substance, such as the active ingredients or one or more excipients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

A “poorly soluble drug” refers to a drug which meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

As used herein, the term “substantially intact” in terms of a specified component, is used herein to mean that the specified component has not been degraded or rendered inactive in an amount less than 5%. The term “essentially intact” is used to represent that less than 2% of the specific component has been degraded or rendered inactive. The term “entirely intact” contains less than 0.1% of the specific component that has been degraded or rendered inactive.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the electromagnetic energy absorbing excipients are adjacent to another atom of the electromagnetic energy absorbing excipients.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature which is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active agent to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Methods and Materials

### A. Materials

Candurin® gold sheen was purchased from EMD Performance Materials (Philadelphia, Pa.). AQOAT® Hypromellose acetate succinate HMP grade (HPMCAS-HMP) was donated by Shin-Etsu Chemical Co., Ltd. (Tokyo, Japan). Boehringer Ingelheim (BI) research compound BI639667 (BI-667) was donated by BI (Ingelheim, Germany). Glass number 50 capillary (2.0 mm) was purchased from Hampton Research Corp. (Aliso Viejo, Calif.). HPLC grade acetonitrile, methanol and Trifluoracetic acid (TFA) were purchased from Fisher Scientific (Pittsburgh, Pa.). Monohydrate and dihydrate sodium phosphate salts were purchased from Fisher Scientific (Pittsburgh, Pa.). Fasted state simulated intestinal fluid (FaSSIF) powder was purchased from Biorelevant.com Ltd (Surrey, United Kingdom).

### B. Characterizing Composition Interactions

1. Modulated Differential Scanning Calorimetry

Modulated differential scanning calorimetry (mDSC) was conducted on a Q20 DSC unit (TA Instruments, New Castle, Del.). 8-10 mg of sample was weighed with a Sartorius 3.6P microbalance (Göttingen, Germany) into standard aluminum pans and covered with a standard aluminum lid. Thermal analysis was performed with a nitrogen sample purge of 50 mL/min. Measurement parameters for detecting changes in melting point (Tₘ) and glass transition (T_{g}) when the EEAE is incorporated were determined using a heating rate of 3°/min from 35-220° C. with a modulation of 0.3° C. every 50 seconds.

2. Fourier-Transform Infrared Spectroscopy

Interaction between the EEAE and other components in the composition were evaluated using Attenuated total reflectance Fourier-transform infrared spectroscopy (ATR-FTIR) on a Nicolet™ iS™ 50 spectrometer (Thermo Scientific, Waltham, Mass.). Measurements were performed using a germanium crystal that supplied constant torque during the analysis. Analysis conditions scanned a range of 700-4000 cm⁻¹ using a resolution of 4 cm⁻¹ with 64 scans. Results were evaluated using OMNIC™ analysis software.

### C. Final Composition Characterization and Performance Evaluation

1. Powder X-Ray Diffraction

Powder X-ray diffraction (PXRD) was performed on a Rigaku MiniFlex600 (Rigaku, The Woodlands, Tx, USA) that utilized a Cu-Ku radiation source operated at a voltage of 40 kV and a current of 15 mA. Powder samples were dispensed in aluminum sample holders. The method parameters for analysis scanned a two-theta range of 10-35° with a scan speed of 2.0°/min, step size of 0.020 while rotating the sample. Data analysis was performed using MDI JADE 9 software (Materials Data Inc., Livermore, Calif.).

2. Wide-Angle X-Ray Scattering

WAXS measurements used a custom-built SAXSLab instrument (SAXSLab, Northampton, Mass., USA) at the University of Texas at Austin (Austin, Tex., USA). The instrument is equipped with a microfocus Cu k-alpha rotating anode X-ray source operated at 50 kV and 0.6 mA and a PILATUS3 R 300K (DECTRIS Ltd., Philadelphia, Pa., USA) detector. The detector is equipped with three detecting modules of 83.8×106.6 mm² sensitive area. The pixel size is 172×172 μm². The distance between the sample and detector ranged from 0.95 to 1.45 m. Disposable glass capillaries (Hampton Research, Aliso Viejo, Calif., USA) of a 2.0 mm outside diameter were used to load samples. Ganesha instrument control center software (SAXSLab, Northampton, Mass., USA) was used to control the instrument. The configuration of 2 apertures WAXS and 2 mm off-centered beam stop was used for all measurements. The acquisition time for each sample was set at 300 s with a beam stop mask and correction for the sample thickness of 2.0 mm. All data were corrected for cosmetic background radiation and an incident beam strength by measuring the X-ray intensity directly on the detector. Data analyses were performed using SAXSGUI software (SAXSLab, Northampton, Mass., USA).

### Example 2—Preparation of Selective Laser Sintering 3D Printing

### A. Printing Parameters

Ritonavir, a thermally labile, shear sensitive, and the poorly water-soluble drug was selected as a model drug. This particular therapeutic agent was tested in U.S. Patent Application No. 2019/037441 and those printing parameters did not lead to the production of a printed tablet or an amorphous solid dispersion. This is further described in Example 3. In particular, the impact of hatch spacing (HS) has not been investigated. When the HS in P1 was increased, the composition was not sintered and the printing failed, see Table 3. U.S. Patent Application No. 2019/037441 has described other printing parameters within its specification that can produce a crystalline tablet, but the HS has not been described. Using an HS of 125, described in P1, and a laser speed of 25 mm/s the printing failed from insufficient energy to sinter. On the other hand, a printed tablet could be formed by increasing the laser speed to 50 mm/s when the HS was decreased to 25. These examples there are different energy thresholds that are required to print a tablet versus converting a tablet to an amorphous state. The total energy applied to the system is a function of the electron laser density and the ability of the composition to absorb a percentage of the energy emitted by the laser. Each composition will have a different electron laser density necessary to overcome each threshold dependent on the composition's capacity to absorb at the wavelength emitted by the laser. In this example, F1-P3-10 had sufficient energy to overcome the threshold to sinter the composition and produce a tablet but not enough energy to convert the tablet to the amorphous form. F1-P4-10 used a higher surface temperature to overcome the second energy threshold needed to melt the crystalline drug, this resulted in an amorphous tablet. Ritonavir's capacity to absorb energy the laser emits allows the F3 formulations to convert to the amorphous phase when higher laser speeds are used (e.g., less electron laser density). Decreased flow properties from the increased ritonavir drug load required the addition of silicon dioxide to improve flow properties to ensure successful printing at the desired layer thickness. The F3 formulations experience a temperature that is greater than ritonavir's melting point in the composition, 122° C., converning it to an amorphous tablet. Considering how sensitive ritonavir is to processing conditions, purity was tested for all formulations with no degradation observed.

TABLE 1
Compositions for the different formulations
used within the printing process.

| Component | F1 (%) | F2 (%) | F3 (%) |
|---|---|---|---|
| Ritonavir | 10 | 20 | 20 |
| Va64 | 87 | 77 | 76 |
| Candurin ® | 3 | 3 | 3 |
| Silicon Dioxide | 0 | 0 | 1 |

TABLE 2
Compositions are shown below as well as
printing parameters used for examples.

| Formulation | % | | S.T. | C.T. | | |
|---|---|---|---|---|---|---|
| Key | RTV | L.S. | (° C.) | (° C.) | H.S. | Comments |
| F1-P1-10 | 10 | 25 | 110 | 90 | 125 | Tablet could |
| | | | | | | not be made |
| F1-P2-10 | 10 | 25 | 110 | 90 | 25 | Tablet could |
| | | | | | | not be made |
| F1-P3-10 | 10 | 25 | 100 | 90 | 25 | Tablet had |
| | | | | | | crystallinity |
| | | | | | | present |
| F1-P4-10 | 10 | 50 | 105 | 90 | 25 | Selected for 10% |
| F2-P1-20 | 20 | 25 | 110 | 90 | 25 | Print failure |
| (without | | | | | | (Poor flow), |
| SiO₂) | | | | | | 1% silicon |
| | | | | | | dioxide added to |
| | | | | | | improve the flow |
| F3-P2S-20 | 20 | 25 | 110 | 90 | 25 | Print failure |
| | | | | | | (Everything |
| | | | | | | melts) |
| F3-P5S-20 | 20 | 50 | 110 | 90 | 25 | Print failure |
| | | | | | | (Tablet sinters |
| | | | | | | to surrounding |
| | | | | | | powder) |
| F3-P6S-20 | 20 | 50 | 105 | 90 | 25 | Print successful |
| F3-P7S-20 | 20 | 75 | 105 | 90 | 25 | Print successful |
| F3-P8S-20 | 20 | 100 | 105 | 90 | 25 | Print successful |

(All formulations have 3% of Candurin ®)

### B. ASD Determination

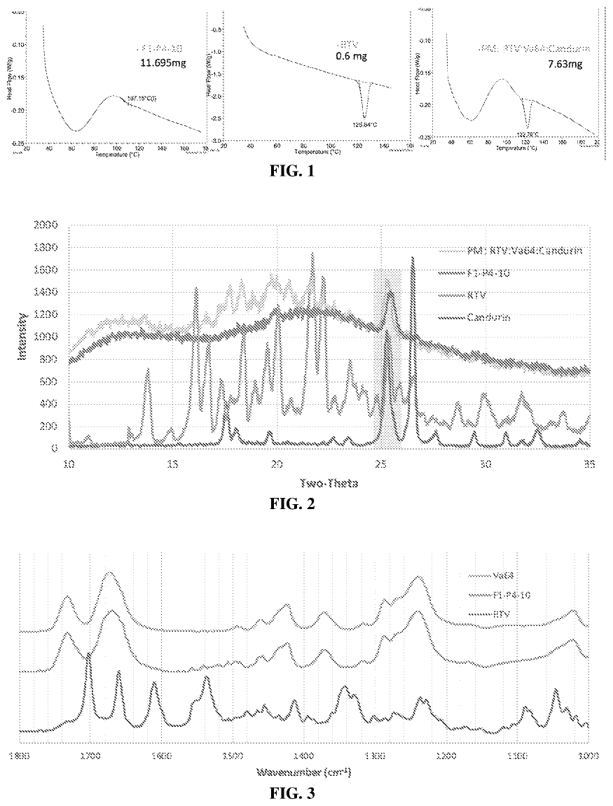

F1-P3-10 exposure to surface temperature, HS and LS were optimized to create a tablet that was converted to the amorphous form. FIGS. 1, 2, 3, & 6 use different solid-state characterization techniques to determine the amorphous nature of the tablet. Final tablets were crushed using a mortar and pestle. Modulated differential scanning calorimetry equilibrated at 35° C. for 5 min, the temperature was then ramped at 3° C./min from 35 to 200° C. with modulation of 0.3° C. every 50 seconds. The absence of a melting endotherm is observed in F1-P4-10. Crystalline RTV would be present at 122° C., the absence of an endotherm suggests the sample is amorphous. Powder X-ray diffraction was performed on a Rigaku MiniFlex600 II instrument equipped with a Cu-Ku radiation source generated at 40 kV and 15 mA. The two-theta angle range, step size, and scan speed were set to 10-35°, 0.02°, and 2°/min, respectively. Candurin® has a very unique diffraction profile across the two-theta region analyzed, with the most intense Bragg's peak being at 19.8 and 25.2 two-theta degrees. These specific peaks associated with Candurin® are also found in the F1-P4-10 sample that was previously shown amorphous by mDSC. In FIG. 2, the region highlighted blue shows the overlap between the amorphous sample and Candurin®. If the presence of Candurin® is disregarded a traditional broad halo would be present. Fourier transform infrared spectroscopy was performed on Attenuated total reflectance Fourier-transform infrared spectroscopy (ATR-FTIR) on a Nicolet™ iS™ 50 spectrometers (Thermo Scientific, Waltham, Mass.). Measurements were performed using a germanium crystal that supplied constant torque during the analysis. Analysis conditions scanned a range of 700-4000 cm⁻¹ using a resolution of 4 cm⁻¹ with 64 scans. Crystalline peaks associated with ritonavir are no longer present in the amorphous, F1-P4-10 sample.

### C. Advanced Technique XRD Determination

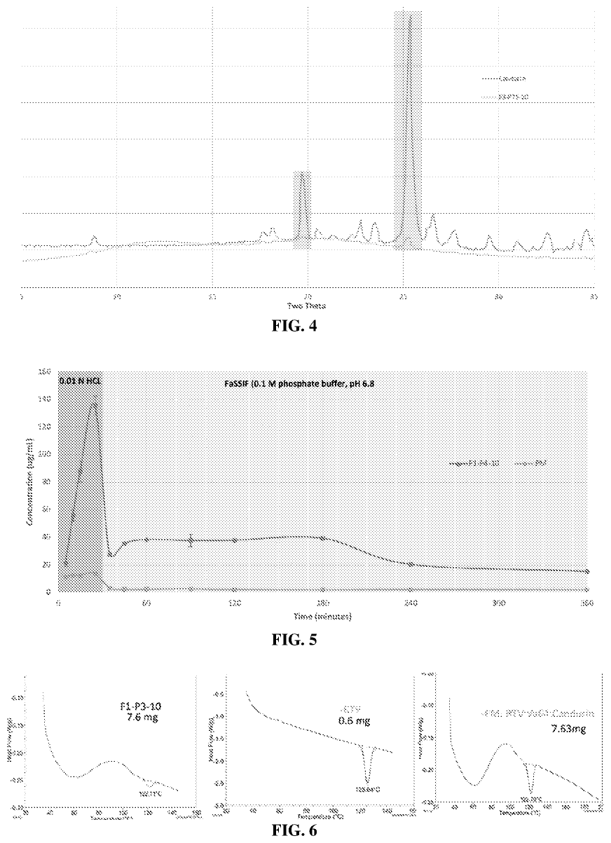

Wide-angle X-ray scattering (WAXS) has been shown to be a sensitive technique being able to detect crystallinity to 0.5% API in composition. This advanced characterization technique was used to ensure crystallinity associated with ritonavir was not present when the drug load increased to 20%. Candurin® has a unique profile that would not be expected to change from the processing method. See FIG. 4. Highlighted in blue are peaks that are associated with Candurin® in the F3-P7S-20 sample. Peaks attributed to ritonavir are not present, indicating an amorphous tablet.

### D. Solubility Enhanced SLS-3DP Ritonavir Formulation

Final Pharmaceutical dosage forms (e.g., SLS-3DP Tablets) dissolution profiles were compared to the physical mixture power. F1-P4-10 tablet weights were: 503.86, 502.78, 520.64 mg. Physical mixture weights were: 500.23, 500.51, and 502.45 mg. The benefit of the printed amorphous solid dispersion was evaluated by a small volume pH shift dissolution with bio-relevant media to mimic gastrointestinal transit of orally administered tablets. Dissolution was performed in an SR8 Plus dissolution tester (Hanson Research Corp., Chatsworth, Calif.) equipped with mini paddles and 150 mL glass vessels operated at a temperature of 37° C. and a paddle speed of 100 rpm. The vessels initially contained 90 mL of 0.01N HCL and at 30 minutes 60 mL of Fassif (2.24 g/L SIF in 0.1 M sodium phosphate buffer, pH 6.8) was added to each vessel to make a total volume of 150 mL. 1 mL samples were taken, immediately filtered through 0.22 um, 13 mm PTFE syringe filters, and diluted 1:1 with methanol. An equivalent amount of media was replaced at all time points: 5, 10, 15, 25, 35, 45, 60, 90, 120, 180, 240, and 360 minutes. The pH was measured at the conclusion of the study to ensure a pH of 6.8 was maintained for all samples. All samples were performed in triplicate (n=3) and ritonavir concentration was determined using the HPLC. A 10-fold concentration increase was seen with F1-P4-10 amorphous tablets compared to the physical mixture in the acidic phase before the pH shift. A 21-fold concentration increase was seen with F1-P4-10 amorphous tablets compared to the physical mixture in the neutral phase at 3 hours. An improved dissolution profile was seen for F1-P4-10 in both the acidic and neutral media. This dissolution profile is shown in FIG. 5.

### Example 3—Reference Examples Fail to Produce Amorphous Compositions

According to the teaching described in U.S. Patent Application No. 2019/037441 that comprises a ternary composition containing a drug, excipient and absorbent material that absorbs electromagnetic radiation at a wavelength emitted by the laser failed to produce an amorphous solid dispersion. Compositions in this example was made according to the parameters and compositions described within the U.S. Patent Application No. 2019/037441 specification. Ritonavir was used as a poorly water-soluble drug, Va64 as a polymer and Candurin®, the absorbing excipient, in a ratio of 10:87:3 by weight, respectively. The printing parameters described in U.S. Patent Application No. 2019/037441 are: Surface temperature (ST) 0-200° C. preferably 70-170° C., Chamber temperature (CT) 25-200° C. preferably 60-150° C., Layer thickness (LT) 10 mm-0.01 mm, beam size (BS) 0.0025-1 mm, scan speed or laser speed (LS) 5 mm/s to 50,000 mm/s preferably 20-300 mm/s, Laser power 0.5 W to 140 W preferably 1.7-8 W, and wavelength 200 nm to 11,000 nm. In this reference example, a surface temperature between 100 and 110° C., chamber temperature of 90° C., the Layer thickness of 0.1 mm, beam size of 0.25 mm, the Laser scan speed of 25 mm/s, Laser power of 2.3 w, and wavelength of 445 nm were used. Table 3 identifies printing parameters used to attempt to create an amorphous solid dispersion from the composition and printing parameters described in U.S. Patent Application No. 2019/037441. This publication fails to describe the hatch spacing which was shown to be useful to create amorphous solid dispersions or render the active pharmaceutical ingredient into an amorphous form. Compositions were made as a tablet, but the tablet was not rendered into an amorphous form nor an amorphous solid dispersion. Instead, crystallinity was detected in the tablet that was formed, F1-P3-10. FIG. 6 confirms the samples contain crystallinity and are not amorphous nor amorphous solid dispersions.

TABLE 3
Compositions made as reference examples from U.S. patent application
No. 2019/037441 are shown below. F1 consists of Ritonavir, Va64,
and Candurin ® in a ratio of 10:87:3 by weight, respectively.

| Formulation | % | | S.T. | C.T. | | |
|---|---|---|---|---|---|---|
| Key | RTV | L.S. | (° C.) | (° C.) | H.S. | Comments |
| F1-P1-10 | 10 | 25 | 110 | 90 | 125 | Tablet could |
| | | | | | | not be made |
| F1-P2-10 | 10 | 25 | 110 | 90 | 25 | Tablet could |
| | | | | | | not be made |
| F1-P3-10 | 10 | 25 | 100 | 90 | 25 | Tablet had |
| | | | | | | crystallinity |
| | | | | | | present |

### Example 4—Analysis and Preparation of Nifedipine Compositions

### A. Experimental Section

i. Materials

The drug, nifedipine, was purchased from Nexconn Pharmtech Ltd. (Shenzhen, China). The polymer, Kollidon® VA64 (average molecular weight 65,000 g/mol), was donated by BASF Corporation (Florham Park, N.J.). The electromagnetic energy-absorbing excipient, Candurin®, was purchased from EMD Performance Materials (Philadelphia, Pa.). Sodium phosphate monobasic, sodium hydroxide, and sodium chloride were purchased from Fisher Scientific (Pittsburgh, Pa.) for buffer preparation. For dissolution, the bio-relevant fasted state simulated intestinal fluid (FaSSIF) powder was purchased from Biorelevant.com LTD (Surrey, United Kingdom). The selective laser sintering 3-Dimensional desktop printer kit was purchased and self-assembled from Sintratec AG (Brugg, Switzerland). HPLC grade methanol and acetonitrile were purchased from Fisher Scientific; all other chemicals and reagents used were ACS grade or higher.

ii. Preliminary Screening and Design of Experiments

The first step of this study was to determine whether nifedipine (NFD) absorbed visible radiation at a wavelength (k) of 455 nm, which corresponded to the wavelength of the visible laser-equipped in the selective laser sintering kit (Sintratec kit, Sintratec, Switzerland). For this purpose, a UV-Visible spectrophotometer was used and the absorption spectra of NFD were evaluated. Further, to understand the relevant processing and formulation parameters, a screening study was conducted, and the preferred parameters were determined. These preferred parameters and their impact were further evaluated using a design of experiments (DoE) approach. This section of the methods discusses the preliminary screening experiments and the DoE used for this study.

iii. UV-Visible Screening Studies

Different NFD concentrations were prepared (20, 40, 80, 160 μg/mL) using methanol as a solvent, and their respective absorbance spectrum was collected using a UV-Visible spectrophotometer (Agilent Cary 8454 UV-Vis Diode Array System, Agilent Technologies, Santa Clara, Calif.). Considering the concentration-based limitations of liquid state quantitative analysis as per Lambert-beer's law, qualitative investigation of NFD was conducted using a UV-vis reflectance probe with a 316L Stainless Steel/Nickel alloy tip and sapphire window, which was developed to analyze the absorbance of solid samples. The prime objective of this study was to observe the absorbance behavior of NFD around 455 nm. For this experiment, the polymer's absorbance was not evaluated, as previous studies have demonstrated that it does not absorb visible radiation.

iv. Parameters Determination

The first step of this study was to determine whether NFD was experimentally absorbing the laser from the source and the laser's impact on the drug molecule. A physical mixture with Kollidon® VA64 and NFD was subjected to a selective laser sintering process at three different conditions, as depicted in Table 4. Post-processing, the printed tablets (printlet) were physically assessed for signs of sintering and were subject to qualitative determination of degradation of the drug post-processing using high-performance liquid chromatography equipped with a mass spectrophotometer (HPLC-MS). Once the laser's impact on the drug was assessed, a preliminary screening study was conducted to determine the formulation and processing parameters. Screening studies were used to determine and set the range of parameters under investigation for optimization studies. Formulations with a 10% w/w NFD drug loading in different concentrations of Candurin® and Kollidon® VA64 were subjected to SLS 3D printing processes with varying processing parameters (surface temperature, chamber temperature, and print speed). Without wishing to be bound by any theory, it is believed that the influence of print parameters (layer height, number of perimeters, perimeter offset, hatching offset, and hatching spacing) was not evaluated as a part of this study and hence were kept constant for all formulations and processing conditions. The formulations and the processing parameters for the screening studies are enlisted in Table 4. For the screening studies, the impact of the parameters on the drug's degradation, amorphous conversion, and, most of all, printability of the drug was assessed. Based on the printability of the printlet the range of the parameters was established for further optimization studies using DoE.

TABLE 4
Formulation composition and printing
parameters for screening studies.

| For- | | | **Processing parameters | | |
|---|---|---|---|---|---|
| mula- | *Formulation parameters | | Surface | Chamber | Laser |
| tion | Candurin ® | Kollidon ® | temperature | temperature | speed |
| no. | (%) | VA64 (%) | (° C.) | (° C.) | (mm/s) |
| S1 | 0 | 90 | 105 | 80 | 50 |
| S2 | 0 | 90 | 105 | 80 | 100 |
| S3 | 0 | 90 | 105 | 80 | 150 |
| S4 | 10 | 80 | 105 | 80 | 50 |
| S5 | 10 | 80 | 105 | 80 | 100 |
| S6 | 10 | 80 | 105 | 80 | 150 |
| S7 | 10 | 80 | 105 | 80 | 200 |
| S8 | 10 | 80 | 105 | 80 | 250 |
| S9 | 15 | 75 | 105 | 80 | 200 |
| S10 | 15 | 75 | 110 | 90 | 250 |
| S11 | 15 | 75 | 110 | 90 | 300 |
| S12 | 30 | 60 | 110 | 90 | 300 |
| S13 | 30 | 60 | 110 | 90 | 400 |
| S14 | 30 | 60 | 115 | 90 | 400 |
| S15 | 30 | 60 | 120 | 100 | 450 |

(*The drug loading in all the formulations was maintained at 10% w/w for the screening studies.
**The print parameters maintained were, layer height: 100 μm; number of perimeters: 1; perimeter offset: 200 μm; hatching offset: 120 μm; hatching spacing: 25 μm)

v. Optimization Studies

After determining the range for the formulation and processing parameters, a response surface DoE study with a 17-run Box-Behnken design was developed using Design-Expert software (Version 10.0.8.0, Stat-Ease, Inc., Minneapolis, Minn., USA) to understand the impact of these parameters on the quality attributes (dimensions, weight variation, hardness, disintegration time, density), stability (% degradation), and crystallinity of the printlet and NFD, respectively. For the design, Candurin® (%), surface temperature (° C.), and laser speed (mm/s) were considered as the independent variables, whereas crystallinity, degradation (%), hardness, average weight (mg), density (mg/cm³), and disintegration time were identified as the dependent variables. The batch to batch variation and reproducibility of the design AM process were assessed by introducing central points in the design, which were repeated five times. The detailed designs and demonstration of variables are shown in FIG. 7 and Table 5.

TABLE 5
Box-Behnken design for the optimization studies.

| | | | B: Surface | C: Laser |
|---|---|---|---|---|
| Run | | A: Candurin ® | temperature | speed |
| no. | Levels | (%) | (° C.) | (mm/s) |
| 1 | −1, 0, 1 | 5 | 110 | 300 |
| 2 | 0, −1, −1 | 10 | 100 | 200 |
| 3 | 1, −1, 0 | 15 | 100 | 250 |
| *4 | 0, 0, 0 | 10 | 110 | 250 |
| *5 | 0, 0, 0 | 10 | 110 | 250 |
| 6 | −1, 1, 0 | 5 | 120 | 250 |
| 7 | 0, 1, −1 | 10 | 120 | 200 |
| 8 | 1, 1, 0 | 15 | 120 | 250 |
| 9 | 1, 0, −1 | 15 | 110 | 200 |
| 10 | 0, 1, 1 | 10 | 120 | 300 |
| *11 | 0, 0, 0 | 10 | 110 | 250 |
| 12 | 1, 0, 1 | 15 | 110 | 300 |
| 13 | −1, −1, 0 | 5 | 100 | 250 |
| 14 | −1, 0, −1 | 5 | 110 | 200 |
| *15 | 0, 0, 0 | 10 | 110 | 250 |
| 16 | 0, −1, 1 | 10 | 100 | 300 |
| *17 | 0, 0, 0 | 10 | 110 | 250 |

(*Represent the five center points in the design)

The drug loading for the optimization studies was set to 5%, although the ratio (wt %) of NFD to Candurin® in the formulation was maintained as per the screening studies.

vi. Feedstock Preparation

Powder-bed-based printers have certain limitations, including but are not limited to the large quantities of feedstock required for the printing process since the powder bed supports the structure being printed. From previous studies without modifying the print bed, typically 150-200 g of feedstock is required based on the dimensions of the printlet, although the un-sintered powder can be recycled. The powder volume can be estimated based on the layer height of the print and the number of layers required to print the part. The second limitation is the absence of mixing of the powder blend during the process. Considering pharmaceutical feedstocks are physical mixtures of multiple components with different densities and bulk properties blended in different ratios, the flow properties of this feedstock play an important role in the quality attributes of the printlet. Physical mixtures containing NFD, Kollidon® VA 64, and Candurin® were prepared using the geometric dilution technique based on the compositions specified in Table 4 and Table 5 for the screening and optimization studies, respectively.

Further, the prepared feedstocks were then passed through the 12-inch diameter, no. 170 sieve (90 μm pore size) to break down any agglomerates present. It should be noted that the sieve pore size should not be more than 100 μm as in that case agglomerates greater than the 100 μm may exist in the feedstock and might be discarded during the printing process instead of being deposited onto the build surface since the layer thickness set for the process is 100 μm. The physical blends were analyzed for drug purity before the process to assess the impact of the process on the degradation of the drug in the blend.

vii. Powder-Bed Fusion Processing (SLS 3D Printing)

The feedstock for each screening formulation or optimization run was exposed to PBF based SLS 3D printing process. This powder batch post sieving was added to the feed region of the benchtop LS 3D printer (Sintratec kit, Sintratec, Switzerland). This SLS printer is equipped with a 2.3W 455 nm blue visible laser. A powder batch of approximately 150 g was used for each build cycle. A CAD file with ten printlet having 5 mm height and 12 mm diameter was loaded onto the Sintratec central software. As mentioned earlier, the print parameters were constant for all print jobs. The layer height, number of perimeters, perimeter offset, hatching offset, hatching spacing was set to 100 μm, 1, 200 μm, 120 μm, and 25 μm, respectively. Furthermore, the processing parameters for the screening conditions and the optimization studies are enlisted in Table 4 and Table 5. For the optimization studies, each manufacturing lot composed of ten printlet, which were tested for their weight, and dimensions using a calibrated weighing balance and a vernier caliper, respectively. Moreover, the tablets from each printed batch were tested for hardness (n=3) (using a TA-XT2 analyzer (Texture Technologies Corp, New York, N.Y., USA)), disintegration time (n=3), crystallinity, and purity (% degradation). The tablets' average dimensions were used to calculate the average volume of tablets for each batch using equation 1, where ‘r’ is the radius and ‘h’ is the height of the tablets. The average volume and average weight of each batch were further used to calculate the tablets' density using equation 2. Density was then used as one of the dependent variables in the DoE for printlet optimization.

$$Volume ( V ) = π {r}^{2} h \qquad 1$$

$$Density ( ρ ) = \frac{mass}{volume} \qquad 2$$

viii. Degradation Testing

As a part of the screening studies after sintering the drug-polymer blend, it was imperative to determine the laser's impact on NFD. For this purpose, high-performance liquid chromatography was used. An analytical technique for the qualitative identification of the degradants was developed for HPLC equipped with a mass spectrophotometer. Moving forward, to quantify the identified degradants, a method for HPLC equipped with a UV-Visible detector was developed.

ix. High-Performance Liquid Chromatography with Mass Spectroscopy (HPLC-MS)

Samples were analyzed using an Agilent 6530 Q-TOF LC/MS with an Agilent Jet Stream electrospray ionization (ESI) source in positive mode. Chromatographic separations were obtained under gradient conditions using an Agilent Eclipse Plus C18 column (50×2.1 mm, 5-micron particle size) with an Agilent Zorbax Eclipse Plus C18 narrow bore guard column (12.5×2.1 mm, 5-micron particle size) on an Agilent 1260 Infinity liquid chromatography system. The mobile phase consisted of eluent A (water+0.1% formic acid) and eluent B (methanol). The gradient was as follows: held at 5% B from 0 to 2 min, 5% B to 20% B from 2 to 5 min, 20% B to 95% B from 5 to 12 min, held at 95% B from 12 to 16 min, 95% B to 5% B from 16 to 16.1 min, and held at 5% B from 16.1 to 20 min. The flow rate was 0.7 mL/min. The sample tray and column compartment were set to 7.5° C. and 30° C., respectively. The fragmentor was set to 80 V. Q-TOF data was processed using Agilent MassHunter Qualitative Analysis software.

x. High-Performance Liquid Chromatography with UV-Visible Detector (HPLC-UV/Vis)

The HPLC method from Ma et al. was adapted and modified to better separate the photolytic degradation experienced in the study. Standards were made using methanol while taking precautions to avoid accidental exposure to light. Using a Dionex UltiMate 3000 high-pressure liquid chromatography (HPLC) system (Thermo Scientific, Sunnyvale, Calif.) equipped with an UltiMate RS Variable Wavelength detector set to 235 nm and Chromeleon 7 software for data acquisition and analysis. During the analysis, the system is held isocratically (70% A: 30% B). The aqueous phase, mobile phase A, consists of HPLC grade water and the organic phase, mobile phase B, consists of acetonitrile. The column separated 10 μL injections with a flow rate of 0.9 mL/min over 30 minutes. A C18, 5×20 mm, 5 um columns (Thermo Scientific, Waltham, Mass.) was used at room temperature to perform the separation.

xi. Printlet Characterization

The crystallinity of the printlet were investigated using X-ray diffraction, and modulated differential scanning (mDSC) analysis, although the mDSC was performed only for the optimized sample. Further, the optimized sample was tested using a pH shift in vitro dissolution test to assess the performance of the printlet in comparison to the crystalline drug.

xii. Powder X-Ray Diffraction Studies (PXRD)

A Rigaku MiniFlex 600 (Rigaku, The Woodlands, Tex.) was utilized to evaluate NFD crystallinity in printed tablets. The instrument is equipped with a Cu-K alpha radiation source. The current is set to 15 mA with a voltage of 40 kV. For sample analysis, the printed tablets are crushed into a fine powder, where the powder is evenly spread into an aluminum sample holder and analyzed over a two theta range of 5-40° 2θ, a scan speed of 2° per minute, and a step size of 0.02° per minute while rotating.

xiii. Modulated Differential Scanning Calorimetry (mDSC)

A Q20 DSC unit (TA Instruments, New Castle, Del.) conducted modulated differential scanning calorimetry (mDSC) measurements at a heating rate of 3° C./min from 35-200° C. During the experiment, the temperature was modulated by 0.3° C. every 50 seconds, with a nitrogen flow of 50 mL/min (Citation of the previous manuscript). For all samples, 8-10 mg was weighed into T-zero pans using a Sartorius 3.6P microbalance (Göttingen, Germany).

xiv. Non-Sink pH-Shift Dissolution

A small-volume, non-sink, pH-shift dissolution evaluated the optimized formulation's solubility enhancement compared to that of the physical mixture. Run 10 floated when placed in the dissolution media and rapidly dissolved by the 10 minute time point. The individual tablet weights for this study were 335.3, 353.5, and 353.6 mg. The weight of the physical mixture used was the same weight of the three tablets, 335.2, 353.7, 353.8 mg. The dissolution media for the study utilized an acidic phase to mimic the stomach, and a neutral phase, to mimic the small intestine. The acidic phase consisted of 0.01 N HCL. The neutral phase consisted of pH 6.8 FaSSIF. A small-volume pH-shift dissolution was performed on an SR8 Plus dissolution tester (Hanson Research Cord., Chatsworth, Calif.) with 150 mL glass vessels and mini-paddles. A paddle speed of 100 RPM was utilized while the temperature was maintained at 37° C. The optimized tablets (n=3) and the Physical mixture (n=3) were dropped into 90 mL of 0.01 N HCL. At 30 minutes, 60 mL of FaSSIF (2.24 g/L SIF in 0.1M sodium phosphate buffer) was used for the pH-shift transition to make a total volume of 150 mL. For all sample pulls, 1 mL of the volume was removed and replaced with an equivalent amount of media. Samples were taken at 5, 10, 15, 25, 35, 45, 60, 90, 120, 180 and 240 minutes. All samples were immediately filtered through a 0.22 um PTFE syringe filter and diluted in 1:1 methanol. Caution was taken to avoid light exposure during the dissolution study by covering the apparatus with aluminum foil to avoid accidental light exposure and keeping overhead lights off when not sampling. Sample concentrations were determined by HPLC analysis using the unmodified method previously mentioned by Ma et al.

xv. Dosage Form Quality Assessment (Dimensions, Microscopy, Hardness, and Disintegration Test)

A VWR® digital caliper (VWR®, PA, U.S.) was used to determine the diameters and thicknesses of the tablets. Images of the printed tablets were taken using Dino-Lite optical microscopy. A texture analyzer (TA-XT2 analyzer, Texture Technologies Corp, New York, USA) along with a one-inch cylinder probe apparatus was used to assess the hardness of the printlet. The test speed was set at 0.3 mm/s and the samples were positioned between the probe across their diameter. The samples' dimensions were inserted in the software before the test, and the probe stopped at a distance of 3 mm from the starting point of the test, which was deemed sufficient to assess the hardness of the samples. The first point of drop-in force (peak force) was recorded as the hardness of the samples and the test was performed in triplicates. The average hardness of each sample was inserted in the DoE to further assess the impact of the independent parameters on the hardness of the tablets. For the disintegration test, a basket-rack assembly filled with 900 mL pH 2 HCl-KCl and maintained at 37±2° C. in a 1000 mL vessel was used. Three tablets were placed in the baskets of the oscillating apparatus, operating at a frequency of 29-32 cycles a minute. The timer was started at the beginning of the test and stopped when the tablets were disintegrated completely with no trace of the samples were observed in the basket. The average disintegration time for each run was recorded and reported as a response parameter in the DoE.

### B. Results

i. Laser Sintering of NFD Promotes Photodegradation and Amorphous Conversion

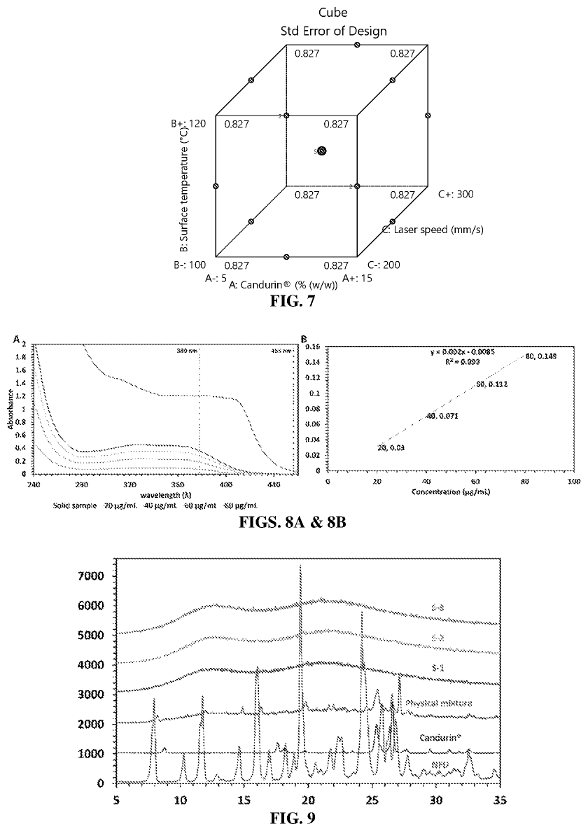

Without wishing to be bound by any theory, it is believed that light-sensitive drugs, absorbing visible radiation at any capacity, will interact with the laser during the SLS process. It was further theorized that if the drug interacted with the laser used in the process, it will undergo photo-lytic degradation, and state transformation (melting). A UV-visible absorption analysis was conducted for NFD in both solid and liquid states to demonstrate the drug's ability to absorb visible radiation. It was observed that NFD, when dissolved in methanol, exhibited considerable absorbance in the visible region (>380 nm). Furthermore, this absorbance in the visible region was also found to be linear, as seen in FIG. 8B, i.e., the absorbance increased with an increase in the concentration of NFD in methanol. This phenomenon described by Lambert-Beer's law has been used in pharmaceutical analysis and for the quantification of drug substances absorbing electromagnetic radiation. Although one limitation of the law is that the linearity fails at higher drug concentration in a solution, where the transmitted radiation is quantified, and absorbed radiation is determined (Mantele and Deniz, 2017). Hence, for the solid crystalline NFD sample, a reflectance probe was used. The solid samples' analysis was qualitative to determine the absorbance spectra of solid NFD samples. It was observed that NFD also absorbed radiation at a wavelength corresponding to that of the laser used in the SLS processing i.e., 455 nm as seen in FIG. 8A.

From the UV-Visible experiments, it was confirmed that NFD absorbs radiation in the visible spectrum; the next step was to observe the laser's impact on NFD post SLS processing. For this purpose, the NFD and Kollidon® VA64 physical mixtures (Formulation S1-S3) without a sintering agent were exposed to the SLS process at three different laser speeds (Table 4). After the process, the printlets were collected, and their morphology was investigated using microscopy to assess if the parts sintered. The physical evaluation and microscopy indicated that the formulations were sintered in the absence of the sintering agent. This can be attributed to the visible radiation-absorbing ability of NFD. The laser power was sufficient for the drug to absorb radiation and undergo solid-liquid-solid state transformation i.e., melting and solidification, which was confirmed by the amorphous nature of NFD in the printlet post-processing (FIG. 9). This melting phenomenon can be attributed to the laser absorption because NFD has a Tₘ of 173±2° C., and the surface temperature was maintained at 105° C. for these formulations, which is significantly below the melting point of NFD and could not have affected the state of the drug.

Further, the printlets (Formulation S1-S3) were predominately degraded upon HPLC analysis (e.g., 92.65% nifedipine degradation), Table 6. HPLC identified two major degradation products (i.e., Peak 4 and Peak 5) and three minor degradation products (i.e., Peaks 1-3). Therefore, nifedipine's degradation mechanism was investigated to make the appropriate formulation and process parameter modifications to minimize degradation.

HPLC-MS studies revealed the molecular composition of the two major degradation products (i.e., peak 4 and peak 5). The molecular structure of the degradation products was determined using the molecular composition and the corresponding double-bond equivalents, FIG. 10. Degradation product 4 results from photolytic degradation caused by visible irradiation of nifedipine; degradation product 5 is from the UV-light mediated oxidation of NFD. For formulations S1-53, degradation products 4 and 5 contribute to more than 70% of the degradation present. The other minor degradation products (i.e., degradation products 1-3) present during HPLC were not detected by LC-MS as they may be nonionizable species; however, it has been reported that other minor degradation products form during photolytic degradation from inter-molecular interactions amongst nifedipine and the intermediates formed (Handa et al., 2014).

ii. Screening Parameters and Range Selection

Screening formulations S4-S8 were prepared with a 1:1 ratio (wt %) of NFD and Candurin®. The degradation of NFD in the presence of Candurin® reduced significantly in formulation S4. The difference can be observed in Table 6, albeit there still was a considerable amount of degradation (≈32%) present in the printlet at a laser speed of 50 mm/s. Before increasing the amount of Candurin® in the formulation, the laser's speed was increased to reduce the time NFD was exposed to the laser source. Increasing the laser speed further reduced the degradation from ≈32% to 26%, 21%, 17%, and finally 10% in formulation S5-S8, respectively, where the laser speed was increased from 50 mm/s (Formulation S4) to 250 mm/s (Formulation S8) at a 50 mm/s increment per formulation. The laser speed was not increased any further as the printlets were brittle and exhibited trace crystallinity by PXRD analysis. Formulations S4-S8 provided valuable information about two of the relevant parameters in these study i.e., presence of Candurin®, and laser speed, where both impacted NFD's degradation, and laser speed also influenced the amorphous conversion.

For further parameter screening the drug-to-Candurin® ratio (wt %) was modified, formulations S9-S11 were prepared with a 1:1.5 NFD and Candurin® ratio (wt %). Formulation S9 was processed at a laser speed of 200 mm/s causing 10% degradation, confirming the continued benefit of Candurin® in the formulation. In comparison, formulation S7, at the same laser speed, observed about 17% degradation. Using a 1:1.5 ratio (wt %) of NFD to Candurin®, the surface temperature for formulation S10 and S11 was increased from 105° C. to 110° C. and the chamber temperature was increased from 80° C. to 90° C. as under the previous temperature conditions formulation S8 was not printable at 250 mm/s. Although formulations S10 (250 mm/s) and S11 (300 mm/s) were printable on increasing the surface temperature, the change in degradation with increasing laser speed was not significant as seen in Table 6. These results point out the impact of surface temperature on the degradation and amorphous conversion of NFD, which was previously not predicted.

Moving forward, the ratio (wt %) of NFD to Candurin® was increased to 1:3 for formulations S12-S15. The degradation observed for formulation S12 was about 5%, which was significantly less compared to S11, which was about 9%. Even though formulation S12 was printable, it was found to have trace crystallinity, and on further increasing the laser speed to 400 mm/s, it was brittle and had about 3% degradation. On increasing the surface temperature to 115° C., 4% degradation was observed with a brittle printlet and trace crystallinity. On further increasing the surface temperature to 120° C. and the laser speed to 450 mm/s, <2% of degradation was observed along with complete amorphous conversion, however, the printlet was found to be brittle.

TABLE 7
Printability and degradation observations
for the screening formulations.

| | | Purity | | | | |
|---|---|---|---|---|---|---|
| Formu- | | (NFD | | | | Print- |
| lation | Amorphous | peak) | Peak 1-3 | Peak 4 | Peak 5 | ability |
| S1 | Yes | 15.46% | 9.14% | 12.28% | 63.11% | Yes |
| S2 | Yes | 7.35% | 5.86% | 9.36% | 77.42% | Yes |
| S3 | Yes | 17.3% | 4.74%% | 8.16% | 69.81 | Yes |
| S4 | Yes | 68.68% | 5.45% | 7.86% | 17.37% | Yes |
| S5 | Yes | 74.59% | 3.51% | 4.26% | 17.65% | Yes |
| S6 | Yes | 79.24% | 2.92% | 4.24% | 12.80 | Yes |
| S7 | Yes | 83.65% | 4.58% | 3.45% | 10.29% | Yes |
| S8 | Crystalline | 90.94% | 2.19% | 1.13% | 5.74% | Brittle |
| S9 | Yes | 90.06 | 1.64% | 3.92% | 4.39% | Yes |
| S10 | Yes | 90.75% | 2.2% | 3.23% | 3.80% | Yes |
| S11 | Yes | 91.4% | 1.89% | 2.72% | 3.98% | Yes |
| S12 | Crystalline | 94.50% | 1.01% | 2.91% | 1.59% | Yes |
| S13 | Crystalline | 97.03% | 0.81% | 1.30% | 0.87% | Brittle |
| S14 | Crystalline | 96.68 | 0.65% | 1.11% | 1.55% | Brittle |
| S15 | Yes | 98.67% | 0.29% | 0.18% | 0.86% | Brittle |

From the results of these screening studies, it was evident that the level of Candurin®, laser speed, and surface temperature play a role in the degradation of NFD. Moreover, laser speed and surface temperature also play a role in the amorphous conversion and printability of the printlet. Hence these three parameters were considered as independent variables for the DoE. Moreover, from the screening studies, the printable range for each of the parameters were selected where Candurin® was used at 1:1, 1:1.5, and 1:3 ratios (wt %) with the drug, the laser speed was set with a minimum of 200 mm/s and a maximum of 300 mm/s, and the surface temperature was set at a minimum of 100° C. and maximum of 120° C.

iii. Optimization Studies

After manufacturing all the formulation compositions using different processing conditions, the manufactured printlets were subjected to various characterization techniques. The data collected from the experiments was introduced as responses to the DoE. Table 8 is a collection of numeric values inserted into the DoE to understand the relationship between each independent variable (Candurin®, laser speed, and surface temperature) on the response variables (crystallinity, purity, hardness, weight, density, disintegration time), which is discussed in-depth in the following sub-sections.

TABLE 8
Compilation of experimental responses for different
combinations of independent variables (Runs 1-17).

| | | | | | Hard- | | | |
|---|---|---|---|---|---|---|---|---|
| | | Diam- | | Den- | ness | DT | | Purity |
| *Run | Height | eter | Weight | sity | (kg) | (sec.) | Crys. | (%) |
| 1 | 6.213 | 11.783 | 296.4 | 0.44 | 0.827 | 6.8 | No | 94.66 |
| 6 | 6.013 | 12.066 | 380.9 | 0.55 | 4.09 | 14.2 | No | 92.41 |
| 13 | 6.361 | 11.412 | 274.5 | 0.42 | 0.368 | 3.78 | Yes | 94.09 |
| 14 | 6.519 | 12.107 | 372.2 | 0.49 | 2.84 | 27 | No | 89.04 |
| 2 | 6.483 | 11.946 | 300.4 | 0.41 | 0.498 | 5 | No | 93.5 |
| 4 | 5.879 | 11.806 | 275.4 | 0.43 | 0.982 | 8.59 | No | 94.09 |
| 5 | 5.879 | 11.806 | 275.4 | 0.43 | 0.982 | 8.77 | No | 94.09 |
| 7 | 6.116 | 12.576 | 406 | 0.53 | 4.68 | 16 | No | 88.62 |
| 10 | 5.829 | 12.223 | 326.5 | 0.47 | 2.88 | 5 | No | 94.85 |
| 11 | 5.879 | 11.806 | 275.4 | 0.42 | 0.982 | 8.4 | No | 94.09 |
| 15 | 5.879 | 11.806 | 275.4 | 0.43 | 0.982 | 8.6 | No | 94.09 |
| 16 | 5.302 | 11.309 | 192.3 | 0.36 | 0.255 | 14.2 | No | 95.55 |
| 17 | 5.879 | 11.806 | 275.4 | 0.43 | 0.982 | 8.4 | No | 94.09 |
| 3 | 5.722 | 10.988 | 210.2 | 0.38 | 0.103 | 7 | No | 94.67 |
| 8 | 5.815 | 11.906 | 311 | 0.48 | 1.54 | 24 | No | 93.81 |
| 9 | 6.008 | 12.078 | 308.3 | 0.4 | 0.785 | 8.6 | No | 92.17 |
| 12 | 5.25 | 11.079 | 224.5 | 0.44 | 0.269 | 2 | No | 96.16 |

(*The runs were randomized to prevent bias)

iv. Crystallinity

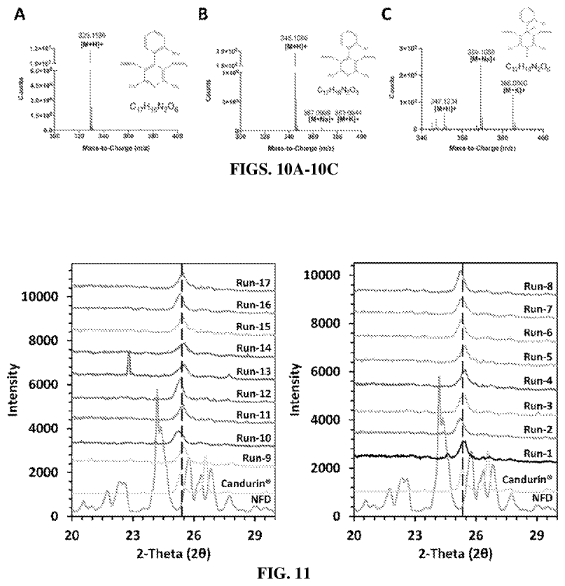

PXRD was used to determine the crystallinity of NFD in the DoE formulation. From the screening studies, increasing the laser speed led to crystallinity or partial amorphous conversion in the formulation. For the DoE samples, the laser speed was maintained at or below 300 mm/s; thereby, it was expected that all the formulations will undergo amorphous conversion and subsequent formation of an amorphous solid dispersion. From the XRD results depicted in FIG. 11, all samples, except for Run 13, demonstrated the absence of crystalline peaks. The two-theta (20) values for these experiments were set from 20-30 degrees as the physical mixture demonstrated strong NFD crystalline peaks in this region.

Moreover, due to the presence of Candurin®, which demonstrated 20 values at 8.9, 17.6, 18.4, 25.3, and 26.5 degrees, it was also included in the overlay created for the analysis. Characteristic NFD peaks can be seen in FIG. 11 at 20 values of 22.4, 24.1, 25.7, 26.6 degrees. The NFD peaks are absent in all the DoE samples except for Run 13, which consisted of 5% Candurin® and was manufactured at a laser speed of 250 mm/s with a surface temperature of 100° C. This may be attributed to the low surface temperature maintained for manufacturing the printlet. In the screening experiments, we observed a relationship between surface temperature and amorphous conversion, where an increase in surface temperature facilitated amorphous conversion as a function of higher energy input. Surface temperature's impact on amorphous conversion was confirmed by observing Run 1 and Run 14, which have similar compositions as Run 13 but were manufactured at a higher surface temperature (110° C.) and Run 1 was processed at a faster laser speed (300 mm/s) than Run 13. Run 3, Run 16 and Run 2 were also manufactured at a surface temperature of 100° C., although they observed complete amorphous conversion. Amongst these runs, Run 3 was processed at the same manufacturing conditions as Run 13 but contained 15% w/w Candurin®. This comparison is interesting as it suggests that Candurin® also plays a role in amorphous conversion and increasing the amount of Candurin® in the formulations facilitates the amorphous conversion of crystalline NFD. Candurin® facilitating amorphous conversion is also seen in Run 16 and Run 2, which have higher amounts (10% w/w) of Candurin® as compared to Run 13 (5% w/w). The peaks which are consistent in all formulations at a 20 value of 25.3 degrees correspond to the Candurin® peaks and should not be mistaken as the presence of crystallinity in the runs.

v. Degradation

Laser-induced degradation was a consideration and parameter for this study. From the screening experiments, the SLS process led to extensive degradation of NFD when no photo-absorbing species, such as Candurin®, were used. It was observed that increasing the ratio (wt %) of Candurin® to NFD reduced the degradation observed in the printlet. Moreover, the screening studies observed the influence of laser speed and surface temperature on NFD degradation, which required further assessment.

Box-Behnken is a frequently used Response Surface Methodology based second-order design alongside 3^{k} factorial and central composite designs (Khuri & Mukhopadhyay, 2010; Czyrski & Sznura, 2019; Wichianphong & Charoenchaitrakool, 2018). Box-Behnken has the advantage of not including all the combinations in which all variables are on the highest or the lowest levels (Politis et al., 2017; Weissman & Anderson, 2015; Zhang et al., 2020). This reduces the number of runs while maintaining the integrity of the design. Moreover, for such optimization studies, preliminary screening experiments to narrow down the minimum and maximum values of the variables is imperative, which was conducted in this study. The use of the Box-Behnken design is popular in industrial research because it is an economical design and requires only three levels for each factor where the settings are −1, 0, 1 (see FIG. 7)³⁰.

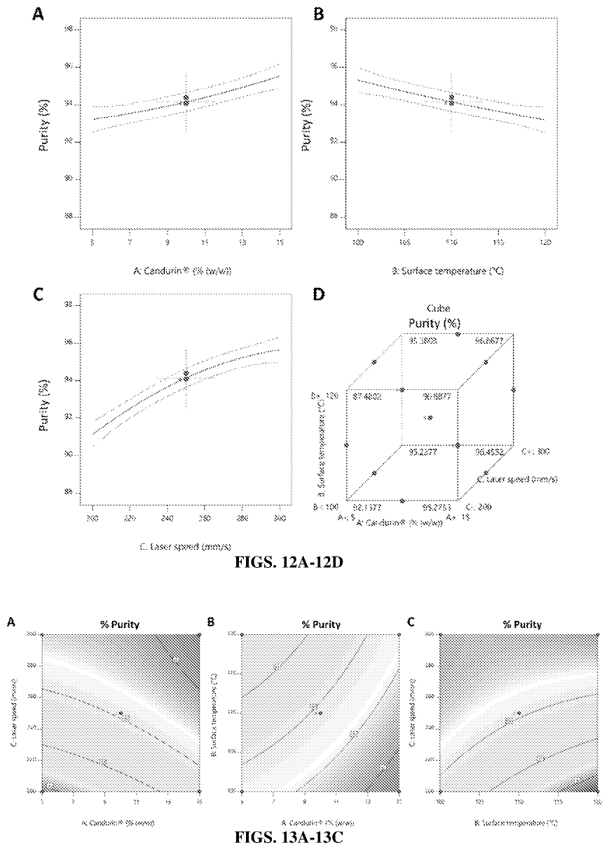

It was observed that multiple interactions occurred between the response and the independent variables after adding responses to the design points; hence the design was fit into a quadratic model. The model was observed to have an F-value of 34.62, which implies the model is significant, and there is only a 0.01% chance that an F-value this large is due to noise. It was also observed that the individual variables, i.e., Candurin® (F=47.43, p=0.0002), surface temperature (F=39.95, p=0.004), and laser speed (F=182.82, p=<0.0001) demonstrated an impact on the degradation of NFD. The impact of these variables was not only significant, but they also demonstrated a correlation with the degradation, which can be seen in FIGS. 12A-12C. The trend that was observed indicates that an increase in the ratio (wt %) of Candurin® to NFD, and an increased laser speed reduce the degradation caused by the process (increase the purity), whereas an increase in surface temperature reduces the purity and increases the degradation observed. This confirms the assumptions made for the laser speed and surface temperature while analyzing the screening formulations.

Furthermore, it was also determined that a combination and interplay between the two processing variables, i.e., surface temperature and laser speed, had a significant impact (F=25.54, p=0.0015) on the purity of the samples. The model suggested that laser speed observed the most significant impact on the degradation of NFD during processing amongst all the independent variables. Laser speed's impact can be observed in FIG. 12D, where the highest purity values correspond to the axes with the highest laser speed i.e., 300 mm/s.

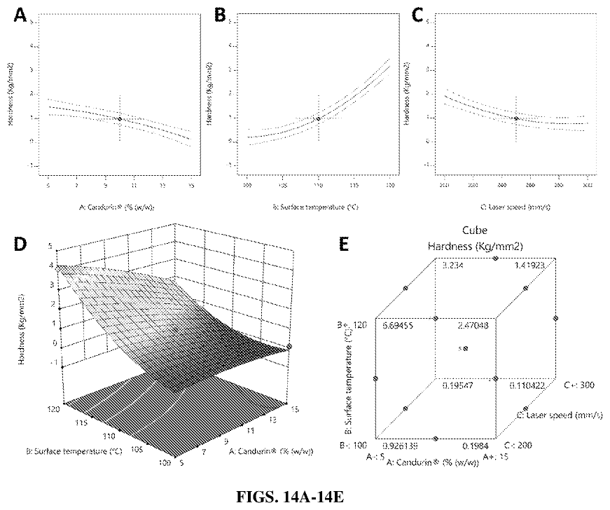

For formulation and process optimization, one parameter is the design's ability to accurately predict change in response to changing a studied variable. This ability can be determined by the ‘Adeq Precision’ of the model, which measures the signal-to-noise ratio (Sabir et al., 2021; Noordin et al., 2004). For this model, a ratio greater than 4 is desirable, and for this design, it was found to be 21.069, which indicates an adequate signal and that this model can be used to navigate the design space. Coefficient estimates or contour lines (FIG. 13) can be used to navigate within the design space. The coefficient estimate represents the expected change in response per unit change in factor value when all remaining factors are held constant. For the tested variables i.e., Candurin®, surface temperature, and Laser speed, the coefficient estimates were found to be 1.16, −1.06, and 2.27 units, respectively. The negative coefficient represents the inverse correlation between surface temperature and purity i.e., purity reduces on increasing surface temperature.

vi. Quality Attributes (Hardness, Density, Weight Variation, and Disintegration Time)

In previous studies, it was observed that different processing parameters demonstrated variability in weight, dimensions, and tensile strength of the printlet. In the previous study, assessing the correlation between the processing parameters and these quality attributes was beyond that study's scope (Davis et al., 2020). The current 17-Run study provided an opportunity to investigate the impact of print speed and surface temperature, along with the formulation composition on these quality attributes.

a. Hardness

The response values for hardness ranged from 0.013 to 4.68 kg/mm² leading to a maximum to minimum response ratio of 45.44. A ratio of more than 10 indicates that a transformation is required; therefore, a square root transformation was performed. The same quadratic model was used because of interactions between independent variables and their impact on the response, as explained in the previous section. The overall model was found to be significant (F=81.95, p=<0.00001). In this case Candurin® (F=104.76, p=<0.0001), surface temperature (F=511.09, p=<0.0001), laser speed (F=67.38, p=<0.0001), Candurin@-Surface temperature (F=10.11, p=0.015) and, Candurin®-laser speed (F=6.86, p=0.03), were found to be significant. The signal-to-noise ratio (32.062) indicated that this model can be used to navigate the design space. The coefficient estimates for all the significant terms, i.e., Candurin®, surface temperature, laser speed, Candurin®-Surface temperature and, Candurin®-laser speed, were −0.2121, 0.6232, −0.2263, −0.1239, and 0.1021 units, respectively. These coefficients indicate that Candurin® and speed have a negative correlation to the hardness of the printlet. This correlation can be seen in FIG. 14A-14C, where an increase in the amount of Candurin® reduces the hardness, and laser speed reduces the hardness of the printlet. In contrast, an increase in the surface temperature increases the hardness of the printlet, which is seen along the axes of the highest value of surface temperature (120° C.) in FIGS. 14D & 14E.

Moreover, the complex interactions between different independent variables on hardness can be observed in the 3D surface plot in FIG. 14. This observed relationship can be explained by the change in the formulation composition from the increase in Candurin® i.e., the amount of Kollidon® VA64 reduces. Candurin® is merely a sintering agent, the sintering occurs due to the thermoplastic nature of Kollidon® VA64 as it absorbs the heat conducted by the sintering agent, undergoes thermal transition, and solidifies, resulting in the sintering of nearby particles together. This data demonstrates that an increase in Candurin® (reduction in Kollidon® VA64) reduces the process's sintering efficacy and leads to brittle structures with low tensile strengths. To see this practically, a direct comparison can be made between Run 6 and Run 8, which are processed at the same conditions (surface temperature: 120° C., laser speed: 250 mm/s), but the former has 5% Candurin® (90% Kollidon® VA64) and latter has 15% Candurin® (80% Kollidon® VA64). Run 6 was found to have a hardness of 4.09 kg/mm², whereas Run 8 had a hardness of 1.54 kg/mm². Further, Run 6 (120° C.) can also be compared to Run 13 (100° C.) to show the impact of surface temperature with other variables constant on the hardness, where Run 13 observed a hardness of 0.368 kg/mm². Additionally, Run 1 (300 mm/s) and Run 14 (200 mm/s) can be used to demonstrate the impact of laser speed when both formulations were processed at 110° C. surface temperature with 5% Candurin® in their formulation and demonstrated a hardness of 0.83 kg/mm² and 2.84 kg/mm²′ respectively.

b. Density and Weight Variation

Weight variability resonates closely to drug content uniformity and dose of the printlets, whereas density relates the dimensions of the printlet to the weight (Lesaffre et al., 2020); hence these two response variables were considered for the evaluation of quality attributes. For both weight and density, the maximum to minimum response ratio was below 10, and hence no transformations were conducted. The data was fit into a quadratic model similar to the above sections. Both weight (F=174.50, p=<0.0001) and density (F=33.80, p=<0.0001) models were found to be significant. All independent variables (Candurin®, surface temperature, laser speed) were found to have an impact on the weight (F=275.94, p=<0.0001; F=756.31 m p=<0.0001; F=456.29, p=<0.0001) and density (F=25.30, p=0.0015; F=215.83, p=<0.0001; F=29.86, p=0.0009) of the printlet. The surface temperature-laser speed impacted the weight of the printlet (F=6.19, p=0.047), whereas Candurin®-laser speed (F=6.57, p=0.0374) had an impact on the density of the printlet. A signal-to-noise ratio of 45.049 and 20.805 was detected for the weight and density responses, suggesting that this model can be used to navigate the design space. Candurin® and laser speed were found to negatively correlate with the weight and the density of the printlet; their coefficients were found to be −33.75, and −43.40 units for weight and −0.02 units for both the variables for density of the printlet. The significance of the coefficients has been explained in the previous sections. The coefficients for surface temperature were positive for both weight (55.88 units) and density (0.058 units), which means an increase in surface temperature increases the tablets' weight and density. These relationships can be observed from the cube and 3D surface plots for weight and density in FIG. 15. The reason behind this trend can be explained by the sintering phenomenon, where a slower laser speed at a higher surface temperature dissipates more energy on the surface as compared to a higher laser speed at a lower surface temperature. This energy causes the thermal conversion of the polymer, leading to an increase in the density of the layer and a reduction in the porosity, which forms a cavity on the print surface during the printing process. The higher the energy dissipation, the steeper the cavity. When the next layer of powder is spread onto this surface, more powder gets filled in the steeper cavity, which gets sintered by the laser, this is responsible for a larger weight of the tablets even though the print dimensions are the same in both these cases.

This can be practically seen in Run 7 where the surface temperature is the maximum (120° C.) and the laser speed set to a minimum (200 mm/s), resulting in a total weight of ≈406 mg, which is the maximum observed weight in this design. Run 7 can be compared with Run 16, which observes a weight of ≈192 mg where the surface temperature is maintained at the minimum value and the laser speed at the maximum value for this design i.e., 100° C. and 300 mm/s. In both these cases, the amount of Candurin® was constant, i.e., 10%. The impact of Candurin® on the weight of the tablets can be assessed by observing Run 6 (5%) and Run 8 (15%) where all the other variables are kept constant (120° C., and 250 mm/s). Run 6 was found to weight ≈380 mg, whereas Run 8 weighted ≈311 mg. This relates to the previously discussed impact of Kollidon® VA64 on the formulation, where the thermal transition of the polymer can increase the hardness of the tablets, which can, in turn, be related to the density of the tablets. All these findings can be used to determine the processing condition and set dimensions in the CAD model for manufacturing dosage forms with a target weight. These trends also help understand the interplay between the processing parameters and the formulation parameters in an SLS 3D printing process.

vii. Characterization of the Optimized Formulation

Though degradation is the key aspect and parameter of this study, the formulations with the lowest degradation levels (i.e., Run 12 and Run 16) were not selected for characterization, as they did not have the best overall printlet characteristics (e.g., hardness). Therefore run 10 was chosen as the optimized formulation for characterization, as these printlets achieved marginally higher degradation (i.e., ˜1%) while having increased printlet hardness. In addition to the characterization reported in Table 4, Run 10 was subject to additional characterization to evaluate the amorphous nature of the printlet further; specifically, if the formulation is miscible and provides solubility enhancement through forming an amorphous solid dispersion.

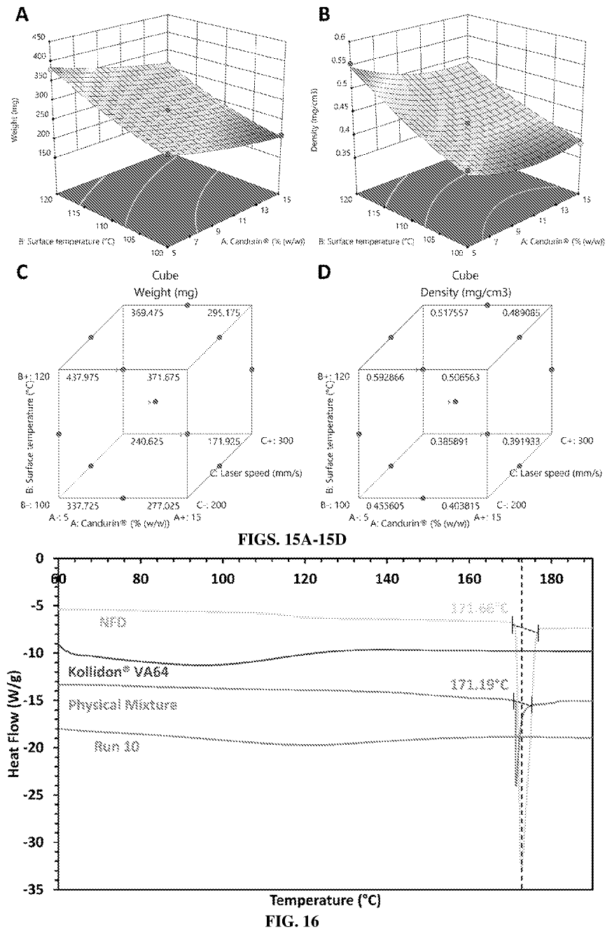

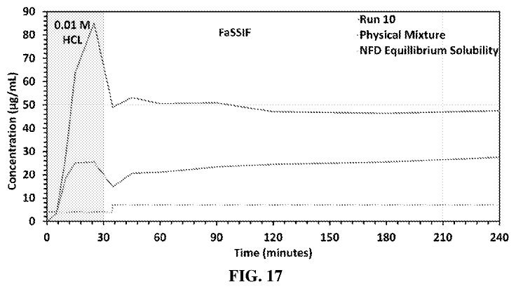

Before evaluating the printlet's solubility enhancement, the miscibility and amorphous nature of the printlet were further investigated. Upon mDSC analysis, the printlet exhibited a single T_{g} onset at 89° C.; the presence of a single T_{g} suggests a miscible formulation with increased stability. The mDSC data also confirmed the prior PXRD characterization, in that, the formulation did not exhibit any melting endotherms, suggesting the absence of crystallinity. The solubility enhancement of the optimized formulation (i.e., Run 10) was evaluated using a non-sink pH-shift small volume dissolution study. See FIG. 16. The optimized formulation achieved a quicker and greater extent of NFD release in the acidic phase, achieving a 21-fold and a 3.4-fold increase in solubility compared to the crystalline NFD and physical mixture before the pH transition, respectively (FIG. 17). Upon pH transition, in the optimized formulation, NFD maintained supersaturation for the study's duration, achieving a 6.7-fold increase and a 1.8-fold increase in solubility compared to the crystalline NFD and physical mixture at the duration of the study.

### C. Discussion

This study demonstrates the utility of a simple pre-formulation UV-Visible absorption experiment to predict a drug's ability to act as an electromagnetic energy-absorbing species during SLS. It was also shown that this laser absorbing activity may lead to electromagnetic radiation-mediated degradation and solid-state transformation of the drug. Although the drug degraded under the influence of the process, it still sintered the drug-polymer physical mixture in the absence of Candurin®. Thereby it can be confirmed that if the drug is stable under the influence of the laser, it can aid the sintering process and reduce the amount or eliminate the need for excipients such as Candurin® in the formulation. In a contrary case where the drug undergoes photolytic degradation, photo absorbing species such as Candurin® that has been used as opacifying agents in the pharmaceutical and cosmeceutical industry can be used to prevent laser mediated degradation. This was demonstrated in the current study for nifedipine, which possesses both π-bonds and non-bonding orbitals (lone pairs in ‘N’ and ‘O’) hence is extremely sensitive to ultraviolet radiation and visible light up to 450 nm. Previous studies have observed that nifedipine gives nitrosophenylpyridine homolog on exposure to daylight, and nitro-phenylpyridine homolog on UV irradiation. This vulnerability towards electromagnetic radiation made NFD an excellent model drug for this study (Hayase et al., 1994).

In an attempt to overcome the degradation that arises when printing NFD:VA64 powder blends, an understanding of the degradation pathway was required to make educated modifications to the process parameters and formulation composition. Therefore, LC-MS was used to determine the molecular formula of the two degradant products identified during the HPLC analysis of the NFD:VA64 printed tablet. NFD undergoes both photolytic degradation and photo-oxidation (Handa et al., 2014; Sadana and Ghogare, 1991; Damian et al., 2007). The molecular formula of the degradants detected by LC-MS i.e., C₁₇H₁₆N₂O₅ (2,6-dimethyl-4-(2-nitrosophenyl)-3,5-pyridinedicarboxylic acid dimethyl ester) also known as NTP and C₁₇H₁₆N₂O₆ (2,6-dimethyl-4-(2-nitrophenyl)-3,5-pyridinecarboxylic acid dimethyl ester) also known as oxidized nifedipine, align with previously reported electromagnetic light-mediated degradation products (Damian et al., 2007; Majeed et al., 1987). Nifedipine on irradiation mainly converts to NTP which is a stable paramagnetic species reported by Damian and colleagues (2006) (Damian et al., 2006). Furthermore, electron paramagnetic spectroscopy (EPR) revealed that an increase in the irradiation time also increased the intensity of the EPR signal, hence the degradation and radical formation were irradiation time dependent (Damian et al., 2006). This helps understand the impact of laser speed on the degradation of NFD. The DoE observed a significant impact of laser speed on the degradation where a higher laser speed (lower exposure time) led to a reduced degradation. Moreover, the kinetics of photo-degradation and photo-oxidation determined by Majeed and colleagues (1987) demonstrated the impact of a variable light source and temperature, where different light sources depicted the different extent of degradation with the highest degradation at 380 nm (Majeed et al., 1987). These findings help understand the impact of the surface temperature on the degradation of NFD as the DoE observed a significant impact of the surface temperature on the extent of degradation of NFD. An increase in temperature led to reduced purity of NFD in the printlet which can be attributed to the lamp placed over the print surface and used as a heat source for SLS printing (Matsuda et al., 1989). The quantum yield for photodegradation is about 0.5; statistically which means that of every two photons absorbed, one causes decomposition of a nifedipine molecule which led to the almost complete degradation of NFD in formulations without Candurin®, whereas on adding Candurin® it coated the NFD crystals and competed with NFD to absorb the electromagnetic energy (Damian et al., 2006). With all other printing parameters unchanged, incorporating Candurin® limited the amount of energy NFD absorbed, and the degradation of NFD was decreased. Moreover, the amount of Candurin® had a significant impact on the purity of NFD in the printlet as shown by the DoE. These findings suggest that SLS processing has some use for processing light-sensitive drugs at this point, as a combination of high laser speed and low surface temperature along with additional formulation considerations, such as the addition of photo-absorbing, opacifying agents is required.

For this study, the transformation of the drug to its amorphous form was important as NFD is a class II drug as per the biopharmaceutical classification system (BCS) and exhibits dissolution limited absorption, and bioavailability (Baghel et al., 2016; Thakkar et al., 2020). Such molecules can be formulated as supersaturating drug delivery systems such as amorphous solid dispersions for an increase in solubility and dissolution rate (Fong et al., 2017). The optimized formulation in this study was found to have a 21-fold increase in solubility as compared to the crystalline NFD before the pH transition and a 6.7-fold increase in solubility after the pH shift. The solubilized drug remains stable at both pH conditions, this trend agrees with previously conducted studies by Theil and colleagues (2016), and Ma and colleagues (2019) that demonstrate solubility enhancement and stability of NFD in Kollidon® VA at the drug load used in the current study. These findings along with the XRD and DSC observations conclude the formation of an ASD post-SLS processing.

Apart from the purity, crystallinity, and performance of the printlets, other quality attributes such as printlet dimensions, tablet weight variation, hardness, and density were also assessed as a part of this study. It was found that the processing and formulation parameters have an influence on these parameters, where an increase in the laser speed, amount of Candurin®, and decrease in surface temperature led to a reduced hardness and average weight of the tablet. Fina and colleagues (2018) observed a similar trend between the laser speed and printlet weight, and hardness where they attributed this to higher energy input from the laser leading to more number necks forming in each layer at lower laser speeds and reduced empty spaces providing more room for powder particles to be sintered thereby creating a heavier printlet¹⁸. However, this relationship was based on observations and only accounted for the impact of the laser which is partially true and can be explained from equation 3:

$${E}_{d} = \frac{n η {P}_{0}}{{V}_{B} {d}_{B}} \qquad 3$$

Where ‘E_{d}’ is the laser power density, ‘n’ are the number of beam passes, ‘η’ is the absorptivity of the material ‘P₀’ (W) is the beam power, ‘V_{B}’ (mm/s) is scanning speed and ‘d_{B}’ (mm) is the beam spot diameter⁴⁴. The equation suggests that the laser power density is inversely proportional to the laser scanning speed and agrees with the observations made by Fina and colleagues (2018). However, the equation does not account for the contribution of surface temperature set on the total energy the surface is exposed to. As per our observations in an SLS process, the heat source exposes the surface of the powder bed to a baseline thermal energy which depends on the set surface temperature, hence the total energy the surface is exposed to is also attributed to the baseline energy from the heat source not just the energy induced by the laser. This was observed in the DoE where an increase in surface temperature led to an increase in printlet density and printlet weight, hence had a similar impact as compared to laser power density. Moreover, as per Equation 3, the absorptivity of the material is directly proportional to the laser power density, so as per the explanation provided by Fina and colleagues (2018) i.e., a higher amount of Candurin® would lead to a higher energy input that should, in turn, lead to an increase in the hardness of the tablets. However, the contrary was observed where an increase in the amount of Candurin® reduces the hardness of the printlet. This is because unlike photo-absorbing polymers such as polyamides (PA-12) designed for SLS printing in the case of pharmaceutical blends where polymers do not absorb the laser directly, photo absorbing species like Candurin® acts as a conducting excipient, which in-turn causes the thermal transition of the polymer, resulting in sintering. Thereby increasing the amount of Candurin® at the cost of Kollidon® VA64 led to a reduction in the hardness and weight of the printlet. These findings add to the current understanding of the SLS process because properties such as weight influence the dose of the printlet, and hardness impacts the stability and performance of the dosage forms.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
U.S. Patent App. Pub. No. 2019/037441
Baghel et al., Journal of Pharmaceutical Sciences, 105:2527-2544, 2016.
Czyrski and Sznura, Scientific Reports, 9:19458, 2019.
Damian et al., Journal of Optoelectronics and Advanced Materials, 9:780, 2007.
Davis et al., Journal of Pharmaceutical Sciences, S0022354920307413, 2020.
Fong et al., Expert Opinion on Drug Delivery, 14:403-426, 2017.
Handa et al., J Pharm Biomed Anal, 89:6-17, 2014.
Hayase et al. J Pharm Sci, 83:532-538, 1994.
Khuri and Mukhopadhyay, WIREs Computational Statistics. 2:128-149, 2010.
Lesaffre et al., Bayesian Methods in Pharmaceutical Research, CRC Press, 2020.
Majeed et al., Journal of Pharmacy and Pharmacology, 39:1044-1046, 1987.
Mantele and Deniz, Spectrochimica Acta Part A: Molecular and Biomolecular Spectroscopy 173:965-968, 2017.
Matsuda et al., International Journal of Pharmaceutics, 54:211-221, 1989.
Noordin et al., Journal of Materials Processing Technology, 145:46-58, 2004.
Politis et al., Drug Development and Industrial Pharmacy, 43:889-901, 2017.
Sabir et al., Journal of Microencapsulation, 0:1-16, 2021.
Sadana & Ghogare, International Journal of Pharmaceutics, 70:195-199, 1991.
Thakkar et al., International Journal of Pharmaceutics, 576:118989, 2020.
Weissman and Anderson, Org. Process Res. Dev., 19:1605-1633, 2015.
Wichianphong and Charoenchaitrakool, Journal of CO2 Utilization, 26:212-220, 2018.
Zhang et al., International Journal of Pharmaceutics 119945, 2020.

What is claimed is:

1. A method of preparing a pharmaceutical composition comprising:
(A) obtaining a composition comprising:
(1) an active pharmaceutical ingredient;
(2) a pharmaceutically acceptable polymer; and
(3) an electromagnetic energy-absorbing excipient;
(B) sintering the composition using a laser in an additive manufacturing process;
to obtain a pharmaceutical composition, wherein the pharmaceutical composition comprises at least 75% of the active pharmaceutical ingredient in the amorphous form.

2. The method of claim 1, wherein the pharmaceutical composition comprises at least 90% of the active pharmaceutical ingredient in the amorphous form.

3. The method of either claim 1 or claim 2, wherein the pharmaceutical composition comprises at least 95% of the active pharmaceutical ingredient in the amorphous form.

4. The method according to any one of claims 1-3, wherein the pharmaceutical composition comprises at least 99% of the active pharmaceutical ingredient in the amorphous form.

5. The method according to any one of claims 1-4, wherein the active pharmaceutical ingredient is present in the pharmaceutical composition as an amorphous solid dispersion.

6. The method according to any one of claims 1-5, wherein the active pharmaceutical ingredient is a poorly soluble drug.

7. The method according to any one of claims 1-6, wherein the active pharmaceutical ingredient is a BCS class 2 drug.

8. The method according to any one of claims 1-6, wherein the active pharmaceutical ingredient is a BCS class 3 drug.

9. The method according to any one of claims 1-6, wherein the active pharmaceutical ingredient is a BCS class 4 drug.

10. The method according to any one of claims 1-9, wherein the active pharmaceutical ingredient is an agent which undergoes degradation at an elevated temperature in a formulation process.

11. The method according to any one of claims 1-10, wherein the active pharmaceutical ingredient is chemically sensitive to temperature.

12. The method according to any one of claims 1-11, wherein the active pharmaceutical ingredient is chemically sensitive to shear.

13. The method according to any one of claims 1-12, wherein the active pharmaceutical ingredient is an agent with a melting point of greater than about 60° C.

14. The method of claim 13, wherein the melting point is from about 60° C. to about 300° C.

15. The method of claim 14, wherein the melting point is from about 80° C. to about 200° C.

16. The method according to any one of claims 1-13, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

17. The method according to any one of claims 1-16, wherein the active pharmaceutical ingredient is an anti-viral agent, antibiotic agent, nonsteroidal anti-inflammatory agent, or heat sensitive agent.

18. The method of claim 17, wherein the anti-viral agent is an anti-retroviral.

19. The method according to any one of claims 1-16, wherein the active pharmaceutical ingredient is an anti-hypertensive agent.

20. The method of claim 19, wherein the anti-hypertensive agent is a calcium channel blocker.

21. The method according to any one of claims 1-20, wherein the pharmaceutical composition comprises from about 1% w/w to about 90% w/w of the active pharmaceutical ingredient.

22. The method according to any one of claims 1-21, wherein the pharmaceutical composition comprises from about 5% w/w to about 50% w/w of the active pharmaceutical ingredient.

23. The method according to any one of claims 1-22, wherein the pharmaceutical composition comprises from about 10% w/w to about 30% w/w of the active pharmaceutical ingredient.

24. The method according to any one of claims 1-22, wherein the pharmaceutical composition comprises from about 5% w/w to about 30% w/w of the active pharmaceutical ingredient.

25. The method according to any one of claims 1-24, wherein the pharmaceutical composition comprises a ratio of the active pharmaceutical ingredient to the electromagnetic energy-absorbing excipient from about 5:1 to about 1:10.

26. The method of claim 25, wherein the ratio is from about 2:1 to about 1:5.

27. The method of claim 26, wherein the ratio is from about 1:1 to about 1:3.

28. The method of claim 27, wherein the ratio is about 1:1, 1:1.5, or 1:3.

29. The method according to any one of claims 1-23, wherein the pharmaceutically acceptable polymer is a cellulosic polymer.

30. The method of claim 29, wherein the cellulosic polymer is a neutral cellulosic polymer.

31. The method of claim 29, wherein the cellulosic polymer is a charged cellulosic polymer.

32. The method according to any one of claims 1-23, wherein the pharmaceutically acceptable polymer is a neutral non-cellulosic polymer.

33. The method of claim 32, wherein the neutral non-cellulosic polymer comprises a poly(vinyl acetate), poly(vinylpyrrolidone), poly(ethylene glycol), poly(ethylene oxide), poly(vinyl alcohol), or methacrylate unit.

34. The method according to any one of claims 1-33, wherein the pharmaceutically acceptable polymer comprises a poly(vinyl acetate) or a methacrylate unit.

35. The method according to any one of claims 1-35, wherein the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

36. The method according to any one of claims 1-35, wherein the pharmaceutical composition comprises from about 5% w/w to about 95% w/w of the pharmaceutically acceptable polymer.

37. The method according to any one of claims 1-36, wherein the pharmaceutical composition comprises from about 50% w/w to about 90% w/w of the pharmaceutically acceptable polymer.

38. The method according to any one of claims 1-37, wherein the pharmaceutical composition comprises from about 60% w/w to about 90% w/w of the pharmaceutically acceptable polymer.

39. The method according to any one of claims 1-38, wherein the electromagnetic energy-absorbing excipient is a material that leads to improved energy absorption.

40. The method according to any one of claims 1-39, wherein the electromagnetic energy-absorbing excipient is a material with a lambda max (λₘₐₓ) equal to the wavelength of the laser.

41. The method of claim 40, wherein the lambda max is from about 50 nm to about 15,000 nm.

42. The method of claim 41, wherein the lambda max is from about 200 nm to about 11,000 nm.

43. The method of claim 40, wherein the lambda max is from about 200 nm to about 1,000 nm.

44. The method according to any one of claims 1-43, wherein the electromagnetic energy-absorbing excipient is an inorganic material.

45. The method according to any one of claims 1-44, wherein the electromagnetic energy-absorbing excipient is an aluminum material.

46. The method of claim 45, wherein the aluminum material is an aluminum inorganic salt.

47. The method of claim 46, wherein the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide.

48. The method of claim 47, wherein the aluminum inorganic salt is potassium aluminum silicate with a coating of titanium dioxide and/or iron oxide.

49. The method of claim 44, wherein the inorganic material is iron oxide, titanium oxide, or silicates.

50. The method according to any one of claims 1-43, wherein the electromagnetic energy-absorbing excipient is an organic material.

51. The method of claim 50, wherein the organic material is a dye.

52. The method of claim 51, wherein the dye is carmine, a phthalocyanine, or a diazo compound.

53. The method according to any one of claims 1-52, wherein the pharmaceutical composition comprises from about 0.01% w/w to about 60% w/w of the electromagnetic energy-absorbing excipient.

54. The method according to any one of claims 1-53, wherein the pharmaceutical composition comprises from about 0.1% w/w to about 50% w/w of the electromagnetic energy-absorbing excipient.

55. The method according to any one of claims 1-54, wherein the pharmaceutical composition comprises from about 1% w/w to about 30% w/w of the electromagnetic energy-absorbing excipient.

56. The method according to any one of claims 1-55, wherein the pharmaceutical composition comprises from about 1% w/w to about 15% w/w of the electromagnetic energy-absorbing excipient.

57. The method according to any one of claims 1-56, wherein the method comprises using a laser with sufficient energy to cause the conversion of the active pharmaceutical ingredient to an amorphous form.

58. The method of claim 57, wherein the method comprises exposing the composition to a laser in a pattern.

59. The method of claim 58, wherein the pattern is prepared by passing the laser over the composition with a laser speed from about 5 mm/s to about 50,000 mm/s.

60. The method of claim 59, wherein the laser speed is from about 10 mm/s to about 1,000 mm/s.

61. The method of claim 60, wherein the laser speed is from about 25 mm/s to about 300 mm/s.

62. The method of claim 61, wherein the laser speed is from about 200 mm/s to about 300 mm/s.

63. The method according to any one of claims 1-62, wherein the laser has a hatch spacing from about 5 mm to about 100 mm.

64. The method of claim 63, wherein the hatch spacing is from about 10 mm to about 50 mm.

65. The method of claim 64, wherein the hatch spacing is from about 10 mm to about 40 mm.

66. The method of claim 65, wherein the hatch spacing is about 25 mm.

67. The method according to any one of claims 1-66, wherein the laser comprises a laser power from about 0.1 W to about 250 W.

68. The method of claim 67, wherein the laser power is from about 0.5 W to about 150 W.

69. The method of claim 68, wherein the laser power is from about 1 W to about 100 W.

70. The method of claim 69, wherein the laser power is from about 1 W to about 10 W.

71. The method according to any one of claims 1-70, wherein the method comprises depositing a layer in a chamber.

72. The method of claim 71, wherein the layer has a layer thickness from about 1 μm to about 100 mm.

73. The method of claim 72, wherein the layer thickness is from about 10 μm to about 10 mm.

74. The method of claim 73, wherein the layer thickness is from about 50 μm to about 1 mm.

75. The method of claim 74, wherein the layer thickness is from 50 μm to about 100 μm.

76. The method according to any one of claims 1-75, wherein the layer comprises a surface temperature at its surface different from a chamber temperature in the chamber.

77. The method of claim 76, wherein the surface temperature is from about 0° C. to about 250° C.

78. The method of claim 77, wherein the surface temperature is from about 50° C. to about 175° C.

79. The method of claim 78, wherein the surface temperature is from about 75° C. to about 150° C.

80. The method of claim 79, wherein the surface temperature is from about 100° C. to about 120° C.

81. The method according to any one of claims 76-80, wherein the chamber temperature is from about 25° C. to about 250° C.

82. The method according to any one of claims 76-81, wherein the chamber temperature is from about 50° C. to about 200° C.

83. The method according to any one of claims 76-82, wherein the chamber temperature is from about 75° C. to about 150° C.

84. The method according to any one of claims 76-83, wherein the surface temperature is more than 15° C. less than the melting point of the composition.

85. The method according to any one of claims 1-84, wherein the laser comprises a beam size from about 0.25 μm to about 1 mm.

86. The method of claim 85, wherein the beam size is from about 1 μm to about 500 μm.

87. The method of claim 86, wherein the beam size is from about 2.5 μm to about 100 μm.

88. The method according to any one of claims 1-87, wherein the laser has a wavelength from about 50 nm to about 15,000 nm.

89. The method of claim 88, wherein the wavelength is from about 200 nm to about 11,000 nm.

90. The method of claim 89, wherein the wavelength is from about 200 nm to about 1,000 nm.

91. The method according to any one of claims 1-90, wherein the laser gives the composition an amount of energy equal to an electron laser density from about 2.5 J/mm³ to about 500 J/mm³.

92. The method of claim 91, wherein the electron laser density is from about 5 J/mm³ to about 250 J/mm³.

93. The method of claim 92, wherein the electron laser density is from about 7.5 J/mm³ to about 50 J/mm³.

94. The method according to any one of claims 91-93, wherein the electron laser density is greater than 2.5 J/mm³.

95. The method according to any one of claims 91-94, wherein the electron laser density is greater than 5 J/mm³.

96. The method according to any one of claims 91-95, wherein the electron laser density is greater than 7.5 J/mm³.

97. The method according to any one of claims 1-96, wherein the composition further comprises one or more excipients.

98. The method of claim 97, wherein the excipient is a processing aid.

99. The method of claim 97 or claim 98, wherein the excipient is an opacifying agent.

100. The method of either claim 97 or claim 98, wherein the excipient is an excipient which improves the flowability of the composition.

101. The method according to any one of claims 97-100, wherein the excipient is a silicon compound.

102. The method according to any one of claims 97-101, wherein the excipient is silicon dioxide.

103. The method according to any one of claims 97-102, wherein the composition comprises from about 0.1% w/w to about 5% w/w of the excipient.

104. The method of claim 103, wherein the composition comprises from about 0.5% w/w to about 2.5% w/w of the excipient.

105. The method of claim 104, wherein the composition comprises from about 0.5% w/w to about 1.5% w/w of the excipient.

106. The method according to any one of claims 1-105, wherein the additive manufacturing technique is selective laser sintering.

107. The method according to any one of claims 1-106, wherein the additive manufacturing technique converts the pharmaceutical composition into a unit dose.

108. The method of claim 107, wherein the unit dose is an oral dosage form.

109. The method of claim 108, wherein the oral dosage form is a tablet.

110. A pharmaceutical composition prepared according to the methods of any one of claims 1-109.

111. A pharmaceutical composition comprising:
(A) an active pharmaceutical ingredient;
(B) a pharmaceutically acceptable polymer; and
(C) an electromagnetic energy-absorbing excipient;
wherein the pharmaceutical comprises at least 75% of the active pharmaceutical ingredient in the amorphous form.

112. The pharmaceutical composition of claim 111, wherein the pharmaceutical composition comprises at least 90% of the active pharmaceutical ingredient in the amorphous form.

113. The pharmaceutical composition of either claim 111 or claim 112, wherein the pharmaceutical composition comprises at least 95% of the active pharmaceutical ingredient in the amorphous form.

114. The pharmaceutical composition according to any one of claims 111-113, wherein the pharmaceutical composition comprises at least 99% of the active pharmaceutical ingredient in the amorphous form.

115. The pharmaceutical composition according to any one of claims 111-114, wherein the active pharmaceutical ingredient is present in the pharmaceutical composition as an amorphous solid dispersion.

116. The pharmaceutical composition according to any one of claims 111-115, wherein the active pharmaceutical ingredient and the pharmaceutically acceptable polymer is homogenously mixed together.

117. The pharmaceutical composition according to any one of claims 111-116, wherein the active pharmaceutical ingredient is a poorly soluble drug.

118. The pharmaceutical composition according to any one of claims 111-117, wherein the active pharmaceutical ingredient is a BCS class 2 drug.

119. The pharmaceutical composition according to any one of claims 111-117, wherein the active pharmaceutical ingredient is a BCS class 3 drug.

120. The pharmaceutical composition according to any one of claims 111-117, wherein the active pharmaceutical ingredient is a BCS class 4 drug.

121. The pharmaceutical composition according to any one of claims 111-120, wherein the active pharmaceutical ingredient is an agent which undergoes degradation at an elevated temperature in a formulation process.

122. The pharmaceutical composition according to any one of claims 111-121, wherein the active pharmaceutical ingredient is chemically sensitive to temperature.

123. The pharmaceutical composition according to any one of claims 111-122, wherein the active pharmaceutical ingredient is chemically sensitive to shear.

124. The pharmaceutical composition according to any one of claims 111-123, wherein the active pharmaceutical ingredient is an agent with a melting point of greater than 60° C.

125. The pharmaceutical composition of claim 124, wherein the melting point is from about 60° C. to about 300° C.

126. The pharmaceutical composition of claim 125, wherein the melting point is from about 80° C. to about 200° C.

127. The pharmaceutical composition according to any one of claims 111-124, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

128. The pharmaceutical composition according to any one of claims 111-127, wherein the active pharmaceutical ingredient is an anti-viral agent, antibiotic agent, nonsteroidal anti-inflammatory agent, or heat sensitive agent.

129. The pharmaceutical composition of claim 128, wherein the anti-viral agent is an anti-retroviral.

130. The pharmaceutical composition according to any one of claims 111-127, wherein the active pharmaceutical ingredient is an anti-hypertensive agent.

131. The pharmaceutical composition of claim 130, wherein the anti-hypertensive agent is a calcium channel blocker.

132. The pharmaceutical composition according to any one of claims 111-131, wherein the pharmaceutical composition comprises from about 1% w/w to about 90% w/w of the active pharmaceutical ingredient.

133. The pharmaceutical composition according to any one of claims 111-132, wherein the pharmaceutical composition comprises from about 5% w/w to about 50% w/w of the active pharmaceutical ingredient.

134. The pharmaceutical composition according to any one of claims 111-133, wherein the pharmaceutical composition comprises from about 10% w/w to about 30% w/w of the active pharmaceutical ingredient.

135. The pharmaceutical composition according to any one of claims 111-133, wherein the pharmaceutical composition comprises from about 5% w/w to about 30% w/w of the active pharmaceutical ingredient.

136. The pharmaceutical composition according to any one of claims 111-135, wherein the pharmaceutical composition comprises a ratio of the active pharmaceutical ingredient to the electromagnetic energy-absorbing excipient from about 5:1 to about 1:10.

137. The pharmaceutical composition of claim 136, wherein the ratio is from about 2:1 to about 1:5.

138. The pharmaceutical composition of claim 137, wherein the ratio is from about 1:1 to about 1:3.

139. The pharmaceutical composition of claim 138, wherein the ratio is about 1:1, 1:1.5, or 1:3.

140. The pharmaceutical composition according to any one of claims 111-139, wherein the pharmaceutically acceptable polymer is a cellulosic polymer.

141. The pharmaceutical composition of claim 140, wherein the cellulosic polymer is a neutral cellulosic polymer.

142. The pharmaceutical composition of claim 140, wherein the cellulosic polymer is a charged cellulosic polymer.

143. The pharmaceutical composition according to any one of claims 111-134, wherein the pharmaceutically acceptable polymer is a neutral non-cellulosic polymer.

144. The pharmaceutical composition of claim 143, wherein the neutral non-cellulosic polymer comprises a poly(vinyl acetate), poly(vinylpyrrolidone), poly(ethylene glycol), poly(ethylene oxide), poly(vinyl alcohol), or methacrylate unit.

145. The pharmaceutical composition according to any one of claims 111-144, wherein the pharmaceutically acceptable polymer comprises a poly(vinyl acetate) or a methacrylate unit.

146. The pharmaceutical composition according to any one of claims 111-146, wherein the pharmaceutically acceptable polymer is a poly(vinyl acetate)-co-poly(vinylpyrrolidone) copolymer, dimethylaminoethyl methacrylate-methacrylic acid ester copolymer, ethylacrylate-methylmethacrylate copolymer, poly(vinyl acetate) phthalate, poly(methacrylate ethylacrylate) (1:1) copolymer, poly(methacrylate methylmethacrylate) (1:1) copolymer, poly(methacrylate methylmethacrylate) (1:2) copolymer, or polyvinyl caprolactam-polyvinyl acetate-polyethylene glycol graft copolymer sodium dodecyl sulfate.

147. The pharmaceutical composition according to any one of claims 111-146, wherein the pharmaceutical composition comprises from about 5% w/w to about 95% w/w of the pharmaceutically acceptable polymer.

148. The pharmaceutical composition according to any one of claims 111-147, wherein the pharmaceutical composition comprises from about 50% w/w to about 90% w/w of the pharmaceutically acceptable polymer.

149. The pharmaceutical composition according to any one of claims 111-148, wherein the pharmaceutical composition comprises from about 60% w/w to about 90% w/w of the pharmaceutically acceptable polymer.

150. The pharmaceutical composition according to any one of claims 111-149, wherein the electromagnetic energy-absorbing excipient is a material that leads to improved energy absorption.

151. The pharmaceutical composition according to any one of claims 111-150, wherein the electromagnetic energy-absorbing excipient is a material with a lambda max (λₘₐₓ) equal to the wavelength of the laser.

152. The pharmaceutical composition of claim 151, wherein the lambda max is from about 50 nm to about 15,000 nm.

153. The pharmaceutical composition of claim 152, wherein the lambda max is from about 200 nm to about 11,000 nm.

154. The pharmaceutical composition of claim 151, wherein the lambda max is from about 200 nm to about 1,000 nm.

155. The pharmaceutical composition according to any one of claims 111-154, wherein the electromagnetic energy-absorbing excipient is an inorganic material.

156. The pharmaceutical composition according to any one of claims 111-155, wherein the electromagnetic energy-absorbing excipient is an aluminum material.

157. The pharmaceutical composition of claim 156, wherein the aluminum material is an aluminum inorganic salt.

158. The pharmaceutical composition of claim 157, wherein the aluminum inorganic salt is bentonite, potassium aluminum silicate, aluminum, aluminum sulfates, sodium aluminum phosphate acidic, sodium aluminum silicate, calcium aluminum silicate, starch aluminum octenyl succinate, or potassium aluminum silicate with a coating of titanium dioxide and/or iron oxide.

159. The pharmaceutical composition of claim 158, wherein the aluminum inorganic salt is potassium aluminum silicate with a coating of titanium dioxide and/or iron oxide.

160. The pharmaceutical composition of claim 155, wherein the inorganic material is iron oxide, titanium oxide, or silicates.

161. The pharmaceutical composition according to any one of claims 111-154, wherein the electromagnetic energy-absorbing excipient is an organic material.

162. The pharmaceutical composition of claim 161, wherein the organic material is a dye.

163. The pharmaceutical composition of claim 162, wherein the dye is carmine, a phthalocyanine, or a diazo compound.

164. The pharmaceutical composition according to any one of claims 111-163, wherein the pharmaceutical composition comprises from about 0.01% w/w to about 60% w/w of the electromagnetic energy-absorbing excipient.

165. The pharmaceutical composition according to any one of claims 111-164, wherein the pharmaceutical composition comprises from about 0.1% w/w to about 50% w/w of the electromagnetic energy-absorbing excipient.

166. The pharmaceutical composition according to any one of claims 111-165, wherein the pharmaceutical composition comprises from about 1% w/w to about 30% w/w of the electromagnetic energy-absorbing excipient.

167. The pharmaceutical composition according to any one of claims 111-166, wherein the pharmaceutical composition comprises from about 1% w/w to about 10% w/w of the electromagnetic energy-absorbing excipient.

168. The pharmaceutical composition according to any one of claims 111-167, wherein the pharmaceutical composition further comprises one or more excipients.

169. The pharmaceutical composition according to any one of claims 111-168, wherein the excipient is a processing aid.

170. The method of claim 168 or claim 169, wherein the excipient is an opacifying agent.

171. The pharmaceutical composition according to any one of claims 111-169, wherein the pharmaceutical composition comprises a flowability excipient.

172. The pharmaceutical composition according to any one of claims 111-171, wherein the flowability excipient is a silicon compound.

173. The pharmaceutical composition according to any one of claims 111-172, wherein the flowability excipient is silicon dioxide.

174. The pharmaceutical composition according to any one of claims 111-173, wherein the composition comprises from about 0.1% w/w to about 5% w/w of the flowability excipient.

175. The pharmaceutical composition of claim 174, wherein the composition comprises from about 0.5% w/w to about 2.5% w/w of the flowability excipient.

176. The pharmaceutical composition of claim 175, wherein the composition comprises from about 0.5% w/w to about 1.5% w/w of the flowability excipient.

177. The pharmaceutical composition according to any one of claims 111-176, wherein the pharmaceutical composition shows an increase in the dissolved concentration of greater than 5 fold compared to a physical mixture at neutral pH.

178. The pharmaceutical composition of claim 177, wherein the increase in dissolved concentration is greater than 10 fold compared to a physical mixture at neutral pH.

179. The pharmaceutical composition according to any one of claims 111-178, wherein the pharmaceutical composition has been processed through an additive manufacturing process.

180. The pharmaceutical composition of claim 179, wherein the additive manufacturing process is selective laser sintering 3D printing.

181. The pharmaceutical composition of either claim 179 or claim 180, wherein the additive manufacturing process is used to produce a unit dose.

182. The pharmaceutical composition of claim 181, wherein the unit dose is an oral dosage form.

183. The pharmaceutical composition of claim 182, wherein the oral dosage form is a tablet.

184. A method of treating or preventing a disease or disorder in a patient comprising administering to the patient in need thereof a therapeutically effective amount of a pharmaceutical composition according to any one of claims 110-183, wherein the active pharmaceutical ingredient is therapeutically effective for the disease or disorder.

185. A pharmaceutical composition comprising:
(A) an active pharmaceutical ingredient; and
(B) an electromagnetic energy-absorbing excipient;
wherein the pharmaceutical comprises at least 75% of the active pharmaceutical ingredient in the amorphous form.

186. A method of preparing a pharmaceutical composition comprising:
(A) obtaining a composition comprising:
(1) an active pharmaceutical ingredient; and
(2) an electromagnetic energy-absorbing excipient;
(B) sintering the composition using a laser in an additive manufacturing process;
to obtain a pharmaceutical composition, wherein the pharmaceutical composition comprises at least 75% of the active pharmaceutical ingredient in the amorphous form.
